(19) United States
(12) Patent Application Publication
(10) Pub. No.: US 2016/0141506 A1
(43) Pub. Date: May 19, 2016

(54) NAPHTHO[2,1-b]FLUORANTHENE COMPOUND, ORGANIC LIGHT-EMITTING DEVICE, DISPLAY APPARATUS, LIGHTING APPARATUS, IMAGE FORMING APPARATUS, AND EXPOSING APPARATUS

(71) Applicant: CANON KABUSHIKI KAISHA, Tokyo (JP)

(72) Inventors: Hirokazu Miyashita, Tokyo (JP); Tetsuya Kosuge, Yokohama-shi (JP); Jun Kamatani, Tokyo (JP)

(21) Appl. No.: 14/939,871

(22) Filed: Nov. 12, 2015

(30) Foreign Application Priority Data
Nov. 14, 2014 (JP) 2014-232194

(51) Int. Cl. H01L 51/00; G03G 15/04; C07C 13/66; H05B 33/08; C07C 13/62; H01L 27/32

(52) U.S. Cl. CPC H01L 51/0056; C07C 13/62; H01L 51/0058; H01L 27/3244; C07C 13/66; H01L 51/0054; H05B 33/0896; G03G 15/04045; H01L 51/5004

(57) ABSTRACT

Aspects of the present invention provide a naphtho[2,1-b]fluoranthene compound represented by the following general formula [1], which has property of high electron injection.

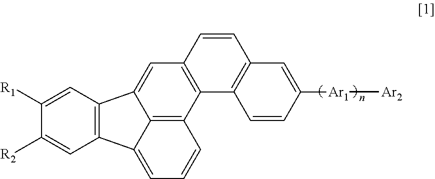

In the formula [1], R₁ and R₂ are respectively selected from the group consisting of hydrogen atom, an alkyl group having 1 to 4 carbon atoms, an alkoxy group having 1 or 2 carbon atom, a halogen atom and a cyano group. Ar₁ denotes a bivalent aromatic hydrocarbon having 6 to 24 carbon atoms. Ar₂ denotes a monovalent aromatic hydrocarbon having 6 to 24 carbon atoms. The integer n is from 0 to 3. In a case where n is 2 or more, each Ar₁ may be the same or different.

### BACKGROUND OF THE INVENTION

1. Field of the Invention

Aspects of the present invention relate to a naphtho[2,1-b]fluoranthene compound, an organic light-emitting device, a display apparatus, a lighting apparatus, an image forming apparatus, and an exposing apparatus.

2. Description of the Related Art

An organic light-emitting device is a device that includes an anode, a cathode, and an organic compound layer placed between the anode and the cathode. Holes and electrons injected from the respective electrodes of the organic light-emitting device are recombined in the organic compound layer to generate excitons and light is emitted as the excitons return to their ground state.

Recent years have seen remarkable advances in the field of organic light-emitting devices. Organic light-emitting devices now feature low driving voltage, various emission wavelengths, rapid response, small thickness, and light-weightiness.

However, there is room for improvement in luminous efficiency and durability. In particular, a lower driving voltage of organic light-emitting device is desired.

A polycondensate aromatic hydrocarbon is used as a material in organic compound layers. Organic light-emitting devices described in Japanese Patent Application Laid Open No. H10-294177 have a structural isomer of naphthofluoranthene as an emitting dorpant. In the above prior art, 4 types of structural isomers of naphthofluoranthene out of 11 possible types are described. The naphthofluoranthene is an example of a polycyclic aromatic hydrocarbon. Herein, the 4 kinds of the structural isomer are shown blow. They are naphtho[2,3-b]fluoranthene, naphtho[1,2-k]fluoranthene, naphtho[2,3-j]fluoranthene, naphtho[2,3-k]fluoranthene.

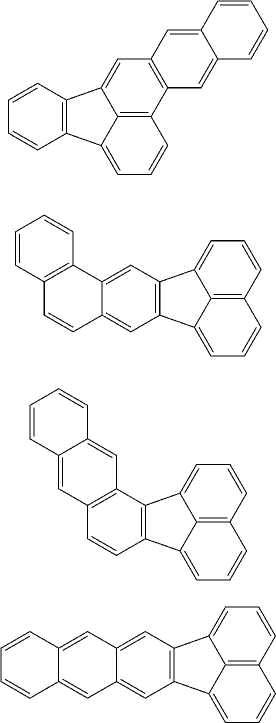

naphtho[2,3-b]naphtho[1,2-naphtho[2,3-naphtho[2,3-fluoranthene k]fluoranthene j]fluoranthene k]fluoranthene

An unsubstituted naphtho[2,1-b]fluoranthene, compound 1032, is described in US Patent Application Publication No. 2004/0076853 as an example of an aggregated compound in an organic light-emitting device which emits excimer emission caused by molecular aggregation. The structure of the naphtho[2,1-b]fluoranthene is shown below.

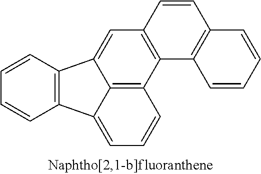 [SMILES: c1ccc2c(c1)-c1cccc3c1c-2cc1ccc2ccccc2c13]

The naphthofluoranthene derivative described in Japanese Patent Application Laid Open No. H10-294177 readily aggregates for the reason that the naphthofluoranthene derivative has a highly planar structure. Also, the HOMO level of the naphthofluoranthene derivative and the LUMO level of the naphthofluoranthene derivative are low. In other words, the ionization potential of the naphthofluoranthene derivative is low and the electron affinity of the naphthofluoranthene derivative is also low. For the above reasons, luminous efficiency of an organic light-emitting device comprising one of the 4 types of the naphthofluoranthene derivative above is low.

The unsubstituted naphtho[2,1-b]fluoranthene described in US Patent Application Publication No. 2004/0076853 readily forms an excimer. This is because a molecule which does not have a substituent cannot decrease its intermolecular interaction. In a case where molecules form an excimer, S1 energy is lower. For this reason, luminous efficiency of an organic light-emitting device having a hole blocking layer comprising an unsubstituted naphtho[2,1-b]fluoranthene is low.

### SUMMARY OF THE INVENTION

An object according to aspects of the present invention is to provide a naphtho[2,1-b]fluoranthene compound having a deep HOMO level, a deep LUMO level, and suppressed molecular aggregation.

Aspects of the present invention provide a naphtho[2,1-b]fluoranthene compound represented by a general formula [1] shown below.

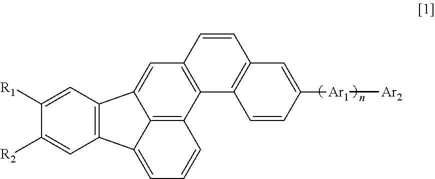

In the formula [1], R₁ and R₂ are independently selected from the group consisting of a hydrogen atom, an alkyl group having 1 to 4 carbon atoms, an alkoxy group having 1 or 2 carbon atom, a halogen atom and a cyano group.

Ar₁ denotes aromatic hydrocarbon having 6 to 24 carbon atoms, and the Ar₁ may be substituted. In a case where the Ar₁ is substituted, the substituent is selected from the group consisting of an alkyl group having 1 to 4 carbon atom, an alkoxy group having 1 or 2 carbon atom, a halogen atom and a cyano group.

Ar₂ denotes aromatic hydrocarbon having 6 to 24 carbon atoms, and the Ar₂ may be substituted. In a case where the Ar₂ is substituted, the substituent is selected from the group consisting of an alkyl group having 1 to 4 carbon atom, an alkoxy group having 1 or 2 carbon atom, a halogen atom and a cyano group.

The character “n” denotes an integer of 0 to 3. In a case where n is 2 or more, each Ar₁ may be the same or different.

Further features of the present invention will become apparent from the following description of exemplary embodiments with reference to the attached drawings.

### BRIEF DESCRIPTION OF THE DRAWINGS

FIG. 1 is a schematic diagram illustrating a spatial structure of a naphtho[2,1-b]fluoranthene ring.

FIG. 2 is a cross-sectional schematic diagram illustrating an organic light emitting element according to aspects of the present invention and a switching element connected to the organic light emitting element.

FIG. 3 is a schematic diagram for describing an example of an image forming device containing the organic light emitting element according to aspects of the present invention.

FIG. 4A and FIG. 4B are schematic diagrams for describing an example of an exposing apparatus containing the organic light emitting element according to aspects of the present invention.

FIG. 5 is a schematic diagram for describing an example of a lighting device containing the organic light emitting element according to aspects of the present invention.

### DESCRIPTION OF THE EMBODIMENTS

According to aspects of the present invention, a naphtho[2,1-b]fluoranthene compound is provided that is represented by the general formula [1]. A chemical structure equivalent to an unsubstituted naphtho[2,1-b]fluoranthene compound is referred to as a naphtho[2,1-b]fluoranthene backbone.

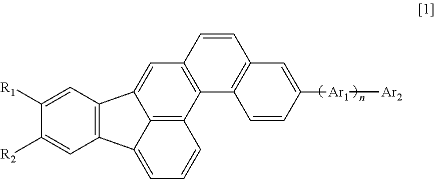

In the formula [1], R₁ and R₂ are independently selected from the group consisting of a hydrogen atom, an alkyl group having 1 to 4 carbon atoms, an alkoxy group having 1 or 2 carbon atom, a halogen atom and a cyano group. R₁ and R₂ are preferably the alkyl group having 1 to 4 carbon atoms.

Specific examples of the alkyl group having 1 to 4 carbon atoms represented by R₁ and R₂ are a methyl group, an ethyl group, an n-propyl group, an iso-propyl group, an n-butyl group, an iso-butyl group, a sec-butyl group and a tert-butyl group.

The alkyl group having 1 to 4 carbon atoms is preferably a methyl group or a tert-butyl group.

Specific examples of the alkoxy group having 1 or 2 carbon atoms represented by R₁ and R₂ are a methoxy group, an ethoxy group.

Specific examples of the halogen atom represented by R₁ and R₂ are fluorine atom, chlorine atom, bromine atom, iodine atom.

In the formula [1], Ar₁ denotes an aromatic hydrocarbon group having 6 to 24 carbon atoms. In one embodiment, an aromatic hydrocarbon is an aromatic group consisting exclusively of carbon atoms and hydrogen atoms, and does not include hetero atoms.

The character “n” denotes an integer of from 0 to 3. In a case where n is 0, Ar₁ is single bond. In case where n is 2 or more, a plurality of Ar₁ are provided, each of which may be the same or different from one another.

Specific examples of the aromatic hydrocarbon represented by Ar₁ include a phenylene group, a biphenyldiyl group, a terphenyldiyl group, a naphthalenedily group, a phenanthrenediyl group, an anthracenediyl group, a benzo[a]anthracenediyl group, a fluorenediyl group, a benzo[a]fluorenediyl group, a benzo[b]fluorenediyl group, a benzo[c]fluorenediyl group, a dibenzo[a,c]fluorenediyl group, a dibenzo[c,g]fluorenediyl group, an acenaphthylenediyl group, a chrysenediyl group, a pyrenediyl group, a benzo[e]pyrenediyl group, a triphenylenediyl group, a benzo[a]triphenylenediyl group, a benzo[b]triphenylenediyl group, a picenediyl group, a fluoranthenediyl group, a benzo[a]fluoranthenediyl group, a benzo[b]fluoranthenediyl group, a benzo[j]fluoranthenediyl group, a benzo[k]fluoranthenediyl group, a perylenediyl group, a naphthacenediyl group, and the like. In one embodiment the aromatic hydrocarbon is a phenylene group, a biphenyldiyl group, a terphenyldiyl group, a naphthalenedily group, a fluorenediyl group, a phenanthrenediyl group, a chrysenediyl group, a fluoranthenediyl group or a triphenylenediyl group.

In the formula [1], Ar₂ denotes a monovalent aromatic hydrocarbon group having 6 to 24 carbon atoms.

Specific examples of the aromatic hydrocarbon represented by Ar₂ include a phenyl group, a biphenylyl group, a terphenylyl group, a naphthyl group, a phenanthryl group, an anthryl group, a benzo[a]anthryl group, a fluorenyl group, a benzo[a]fluorenyl group, a benzo[b]fluorenyl group, a benzo[c]fluorenyl group, a dibenzo[b,h]fluorenyl group, a dibenzo[c,g]fluorenyl group, an acenaphtylenyl group, a chrysenyl group, a benzo[b]chyrysenyl group, a pyrenyl group, a benzo[e]pyrenyl group, a triphenylenyl group, a benzo[a]triphenylenyl group, a benzo[b]triphenylenyl group, a picenyl group, a fluoranthenyl group, a benzo[a]fluoranthenyl group, a benzo[b]fluoranthenyl group, a benzo[j]fluoranthenyl group, a benzo[k]fluoranthenyl group, a perylenyl group, a tetracenyl group, and the like. In one embodiment the aromatic hydrocarbon is a phenyl group, a biphenylyl group, a terphenylyl group, a naphthyl group, a fluorenyl group, a phenanthryl group, a chrycenyl group, a fluoranthenyl group, or a triphenyl group.

It is noted that, according to one embodiment, the aromatic hydrocarbon represented by the Ar₁ or the Ar₂ may have a substituent. In a case where the aromatic hydrocarbon is substituted, the substituent is selected from the group consisting of an alkyl group having 1 to 4 carbon atoms, an alkoxy group having 1 or 2 carbon atoms, a halogen atom and a cyano group.

In one embodiment, the alkyl group having 1 to 4 carbon atoms provided as a substituent is the same as that of the alkyl group having 1 to 4 carbon atoms represented by R₁ or R₂. For example, the alkyl group may be a methyl group or a tert-butyl group. An example of the alkoxy group having 1 or 2 carbon atoms is a methoxy group or an ethoxy group.

In the formula [1], n denotes integer of from 0 to 3. For example, n can denote 0 or 1, and in one embodiment n denotes 1. Herein, in a case where n denotes 2 or more, a plurality of Ar₁ may be provided that are each the same or different from one another. And in a case where n denotes 0, Ar₂ is directly bound to the naphtho[2,1-b]fluoranthene backbone.

In the formula [1], according to one embodiment, both of R₁ and R₂ of the naphtho[2,1-b]fluoranthene compound are hydrogen atoms.

In addition, in the formula [1], according to one embodiment, Ar₁ of the naphtho[2,1-b]fluoranthene compound is selected from the group consisting of a phenylene group, a naphthalenediyl group, a fluorenediyl group, a phenanthrenediyl group, a chrysenediyl group, a biphenyldiyl group, a terphenyldiyl group, a fluoranthenediyl group and a triphenylenediyl group.

In the formula [1], according to one embodiment, Ar2 of the naphtho[2,1-b]fluoranthene compound is selected from the group consisting of a phenyl group, a naphthyl group, a fluorenyl group, a phenanthryl group, a chrysenyl group, a biphenylyl group, a terphenylyl group, a fluoranthenyl group, a triphenylenyl group.

In addition, in the formula [1], according to one embodiment, Ar₁ of the naphtho[2,1-b]fluoranthene compound is selected from the group consisting of a phenylene group bound to the naphtho[2,1-b]fluoranthene backbone, with Ar₂ at the 1-position and the 4-position, a naphthalenediyl group bound to the naphtho[2,1-b]fluoranthene backbone, with Ar₂ at the 2-position and the 6-position, a 9,9-dimethyl fluorenediyl group bound to the naphtho[2,1-b]fluoranthene backbone, with Ar₂ at the 2-position and the 7-position, a phenanthrenediyl group bound to the naphtho[2,1-b]fluoranthene backbone, with Ar₂ at the 2-position and the 7-position, a biphenyldiyl group bound to the naphtho[2,1-b]fluoranthene backbone, with Ar₂ at the 4-position and the 4′-position, and a p-terphenyldiyl group bound to the naphtho[2,1-b]fluoranthene backbone, with Ar₂ at the 4-position and the 4″-position. According to one embodiment, the Ar₂ bound to the naphtho[2,1-b]fluoranthene compound is selected from the group consisting of a phenyl group bound at the 1-position, a naphthyl group bound at the 2-position, a 9,9-dimethyl fluorenyl group bound at the 2-position, a phenanthryl group bound at the 2-position, a biphenylyl group bound at the 4-position, p-terphenylyl group bound at the 4-position, a fluoranthenyl group bound at the 3-position or the 8-position, and a triphenylenyl group bound at the 2-position.

### Properties of the naphtho[2,1-b]fluoranthene compound

In the field of an organic light-emitting device comprising a pair of electrodes and a light emitting layer placed between the pair of electrodes, it is known that luminous efficiency of an organic light-emitting device can be increased by placing a hole blocking layer at the cathode side of the light emitting layer. In addition, for decreasing a driving voltage, the hole blocking layer preferably has both of a property of electron injection from a cathode, an electron injection layer, or an electron transport layer, and a property of electron injection to a light emitting layer.

Therefore, the following 4 points are required for a material for the hole blocking layer. Herein, a deep HOMO level or a deep LUMO level indicates a low HOMO level or a low LUMO level. In other words, the low HOMO level or the low LUMO level is far below the vacuum level and an absolute value of the HOMO level or an absolute value of LUMO level is higher.

(1) Deep HOMO level

In a case where HOMO level of a light emitting layer is lower than HOMO level of a hole blocking layer, suppression of a hole transfer from the light emitting layer to the hole blocking layer can be reduced. This is because a barrier from the light emitting layer to the hole blocking layer is high. As a result, the luminous efficiency of the organic light-emitting device is increased. HOMO level of the hole blocking layer is preferably higher than HOMO level of the light emitting layer by 0.3 eV or more, and more preferably by 0.5 or more.

It is noted that the HOMO level of a light emitting layer refers to the HOMO level of a compound with the maximum weight ratio among the compounds constituting the light emitting layer. The same applies to the HOMO level of a hole blocking layer.

### (2) Large S1 Energy

In a case where S1 energy of a hole blocking layer is sufficiently larger than S1 energy of a light emitting layer, it is possible that excitons are prevented from transferring from a light emitting layer to a hole blocking layer. This enables the excitons to stay in the light emitting layer and results in increasing luminous efficiency of the organic light-emitting device. It is noted that S1 energy of the light emitting layer refers to the S1 energy of a compound with the maximum weight ratio among the compounds constituting the light emitting layer.

### (3) Non-Planer Molecular Plane

A non-planer molecular is inhibited from forming an excimer. The S1 energy of a molecular forming an excimer is smaller than S1 energy of a molecular that does not form an excimer. As a result, that the transfer of excitons from a light emitting layer to a hole blocking layer is not possible. Luminous efficiency of the organic light-emitting device can be increases by suppressing the formation of the excimer and the exciton transfer from the light emitting layer to the hole blocking layer.

### (4) Deep LUMO Level

A case where a barrier between LUMO level of a hole blocking layer and energy level of a cathode or LUMO level of a layer placed at the cathode side of the hole blocking layer is small can be advantageous in that it may be easy to transfer electrons to the hole blocking layer. A difference between LUMO level of the hole blocking layer and LUMO level of the layer placed at the cathode side of the hole blocking layer may be 0.5 eV or less, and more preferably 0.3 eV or less.

Properties for naphthofluoranthene backbones having the above characteristics were calculated by using a molecular orbital calculation. Table 1 below shows the planarity of rings for the optimized structure, the S1 energy, the calculated values of HOMO level, and the calculated values of LUMO level. It is noted that the molecular orbital calculation is executed by density-functional approach at B3LYP/6-31G* level.

TABLE 1

| | | | Wavelength | | |
|---|---|---|---|---|---|
| | | | conversion | | |
| | | | value of | HOMO | LUMO |
| | Structural | Plana- | S1 | value/ | value/ |
| | formula | rity | energy/nm | eV | eV |
| Naphtho[2,1- b] Fluoranthene | 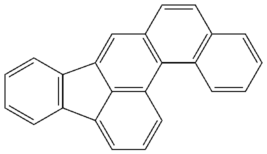 [SMILES: c1ccc2c(c1)-c1cccc3c1c-2cc1ccc2ccccc2c13] | Non- planar | 386 | −5.58 | −1.87 |
| Naphtho[2,3- b] fluoranthene | 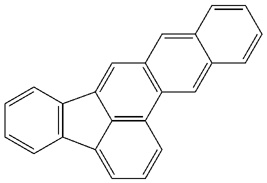 [SMILES: c1ccc2c(c1)-c1cccc3c1c-2cc1cc2ccccc2cc13] | Planar | 397 | −5.21 | −1.79 |
| Naphtho[1,2- k] fluoranthene | 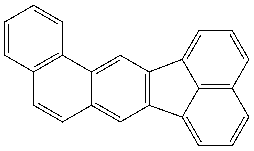 [SMILES: c1cc2c3c(cccc3c1)-c1cc3c(ccc4ccccc43)cc1-2] | Planar | 399 | −5.26 | −1.74 |
| Naphtho[2,3- j] fluoranthene | 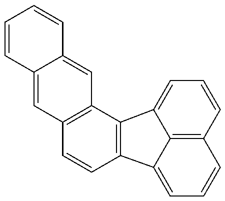 [SMILES: c1ccc2cc3c4c(ccc3cc2c1)-c1cccc2cccc-4c12] | Planar | 561 | −4.81 | −2.13 |
| Naphtho[2,3- k] fluoranthene | 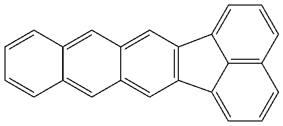 [SMILES: c1ccc2cc3cc4c(cc3cc2c1)-c1cccc2cccc-4c12] | Planar | 439 | −4.78 | −1.63 |
| Naphtho[1,2- b] fluoranthene | 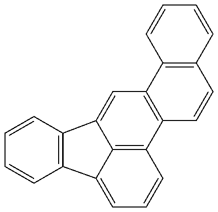 [SMILES: c1ccc2c(c1)-c1cccc3c1c-2cc1c2ccccc2ccc31] | Planar | 381 | −5.42 | −1.76 |
| Naphtho[2,1- j] fluoranthene | 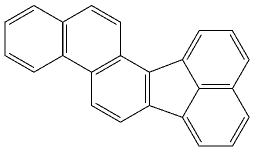 [SMILES: c1cc2c3c(cccc3c1)-c1c-2ccc2c1ccc1ccccc12] | Planar | 432 | −5.35 | −1.90 |
| Naphtho[1,2- j] fluoranthene | 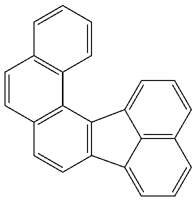 [SMILES: c1cc2c3c(cccc3c1)-c1c-2ccc2ccc3ccccc3c12] | Non- planar | 450 | −5.27 | −1.91 |
| Naphtho[2,1- a] fluoranthene | 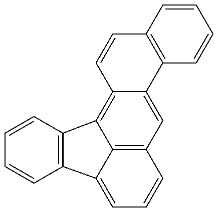 [SMILES: c1ccc2c(c1)-c1cccc3cc4c(ccc5ccccc54)c-2c13] | Planar | 442 | −5.26 | −2.08 |
| Naphtho[1,2- a] fluoranthene | 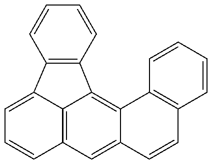 [SMILES: c1ccc2c(c1)-c1cccc3cc4ccc5ccccc5c4c-2c13] | Non- planar | 459 | −5.22 | −2.08 |
| Naphtho[2,3- a] fluoranthene | 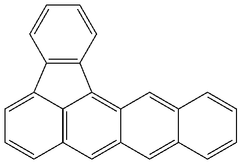 [SMILES: c1ccc2c(c1)-c1cccc3cc4cc5ccccc5cc4c-2c13] | Planar | 622 | −4.75 | −2.46 |

Firstly, the significance of the HOMO level is explained. It is preferable that the HOMO level of a material for a hole blocking layer be deeper than the HOMO level of a material for a light emitting layer. The HOMO level of the naphtho[2,1-b]fluoranthene according to aspects of the present invention is estimated −5.58 eV, which is the deepest in the naphthofluoranthene backbone compounds shown in Table 1. Thus, the naphthofluoranthene backbone is excellent in terms of suppressing a hole leakage from the light emitting layer and in view of point (1), the naphthofluoranthene according to aspects of the present invention is excellent as a hole blocking material.

Next, the significance of the S1 energy is explained. The S1 energy of a hole blocking material is preferably larger than the S1 energy of a light emitting material. The S1 energy of the naphtho[2,1-b]fluoranthene compound according to aspects of the present invention is estimated at 385 nm, which is a sufficiently large value. In a case where the S1 energy is less than 400 nm when converted to wavelength, the naphtho[2,1-b]fluoranthene is excellent in suppressing an exciton leakage from a light emitting layer. Therefore, in view of point (2) as a hole blocking material, the naphthofluoranthene according to aspects of the present invention is more preferable.

Next, the significance of planarity of the naphthofluoranthene backbone is explained. Planarity of molecule is estimated by dihedral angle between a ring of a compound and other ring of the compound. “High planarity” means that the dihedral angle is large, “low planarity” means that the dihedral angle is small. Planarity of a molecule relates to interaction of molecules. The larger the planarity of a molecule is, the larger the interaction between molecules is, and thus a stronger stack of molecules can be formed. Because of this, in a case where the planarity of a molecule is low, the generation of excimer of which S1 value is small or the like can result. Also, a property of suppressing exciton leakage from the light emitting layer may be low, which is not preferable.

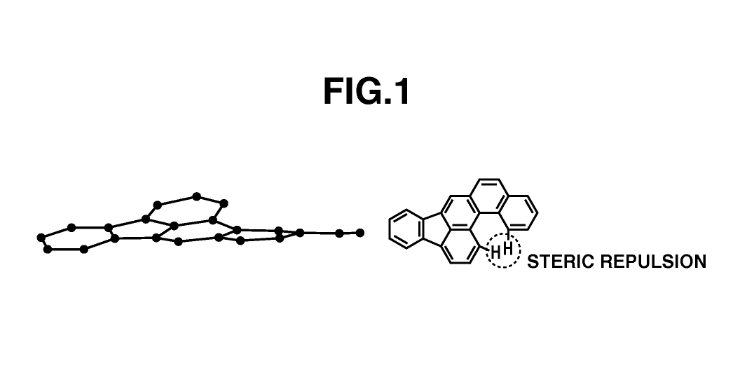

As the planarity of the naphtho[2,1-b]fluoranthene backbone according to aspects of the present invention is not planar, the naphtho[2,1-b]fluoranthene backbone is able to suppress generation of an excimer. Also, the property of suppressing exciton leakage from the light emitting layer is superior. Namely, in view of point (3), the naphthofluoranthene according to aspects of the present invention is more preferable as a hole blocking material. Herein, the term “non-planar” denotes that, for example a shown in FIG. 1, a ring of naphtho[2,1-b]fluoranthene is twisted by steric repulsion between the hydrogen atom at the 1 position of naphtho[2,1-b]fluoranthene and the hydrogen atom at the 14 position of naphtho[2,1-b]fluoranthene. It is noted that in FIG. 1, the hydrogen atoms are omitted for clarity in viewing the ring distortion.

Lastly, significance of the LUMO level is explained. It is preferable that the LUMO level of a hole blocking material is sufficiently deep, and the difference between LUMO level of a hole blocking material and LUMO level of a layer adjacent to a cathode or a hole blocking layer is small. The LUMO level of the naphtho[2,1-b]fluoranthene compound is estimated at −1.87 eV. Also, the property of electron injection from a cathode, a electron injection layer, or a electron transport layer is high. Namely, in view of point (4), the naphtho[2,1-b]fluoranthene compound according to aspects of the present invention is preferable as a hole blocking material.

From the above, the naphtho[2,1-b]fluoranthene compound according to aspects of the present invention satisfies the above points (1) to (4). On the other hand, the other 10 types of naphthofluoranthene compound isomers do not satisfy all of the points (1) to (4).

Here, the backbone of the compound is significant partial structure in the compound. The partial structure mainly influences physical property values such as the S1 energy, the HOMO level and the LUMO level of the compound thereof. In contrast, the R₁ and R₂ of the compound can be used for minor adjustments of physical property values of the compound.

From the above, it can be seen that the naphtho[2,1-b]fluoranthene backbone is the most preferable among all types of naphthofluoranthene backbones as a compound for a hole blocking material.

On the other hand, the naphtho[2,1-b]fluoranthene compound according to aspects of the present invention has an aromatic hydrocarbon group at the 3 position as a substituent. There are 14 possible points for substituent positions on the naphtho[2,1-b]fluoranthene backbone, as shown in the following chemical formula.

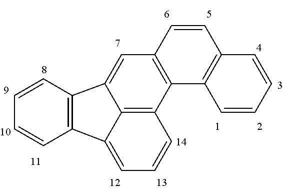 [SMILES: c1ccc2c(c1)-c1cccc3c1c-2cc1ccc2ccccc2c13]

For a substitution position at the 14 points of the naphtho[2,1-b]fluoranthene backbone, molecular orbital calculations were performed for the compound with a phenyl group substituted as an aromatic hydrocarbon group, and a dihedral angle between a benzene ring and naphtho[2,1-b]fluoranthene ring were compared.

TABLE 2

| | Structural | dihedral |
|---|---|---|
| | formula | angle |
| Ph at 1 position | 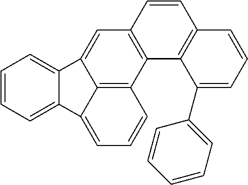 [SMILES: c1ccc(-c2cccc3ccc4cc5c6c(cccc6c4c23)-c2ccccc2-5)cc1] | 42.9 |
| Ph at 2 position | 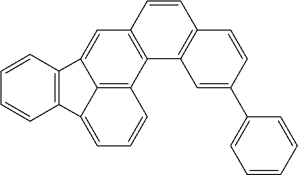 [SMILES: c1ccc(-c2ccc3ccc4cc5c6c(cccc6c4c3c2)-c2ccccc2-5)cc1] | 38.6 |
| Ph at 3 position | 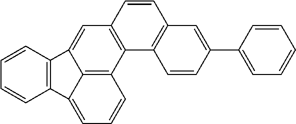 [SMILES: c1ccc(-c2ccc3c(ccc4cc5c6c(cccc6c43)-c3ccccc3-5)c2)cc1] | 35.7 |
| Ph at 4 position | 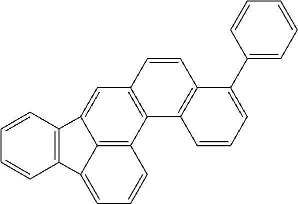 [SMILES: c1ccc(-c2cccc3c2ccc2cc4c5c(cccc5c23)-c2ccccc2-4)cc1] | 56.4 |
| Ph at 5 position | 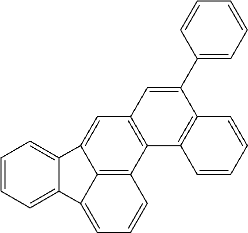 [SMILES: c1ccc(-c2cc3cc4c5c(cccc5c3c3ccccc23)-c2ccccc2-4)cc1] | 59.2 |
| Ph at 6 position | 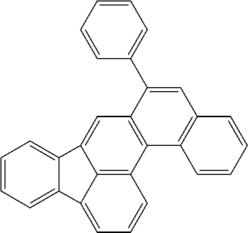 [SMILES: c1ccc(-c2cc3ccccc3c3c2cc2c4c(cccc43)-c3ccccc3-2)cc1] | 56.9 |
| Ph at 7 position | 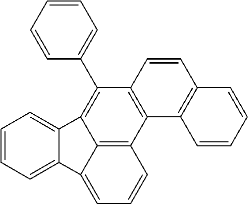 [SMILES: c1ccc(-c2c3c4c(cccc4c4c2ccc2ccccc24)-c2ccccc2-3)cc1] | 79.9 |
| Ph at 8 position | 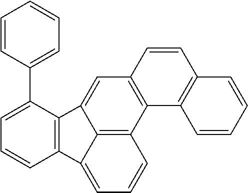 [SMILES: c1ccc(-c2cccc3c2-c2cc4ccc5ccccc5c4c4cccc-3c24)cc1] | 57.4 |
| Ph at 9 position | 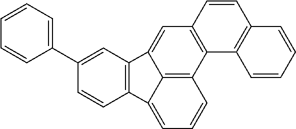 [SMILES: c1ccc(-c2ccc3c(c2)-c2cc4ccc5ccccc5c4c4cccc-3c24)cc1] | 38.0 |
| Ph at 10 position | 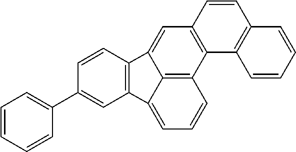 [SMILES: c1ccc(-c2ccc3c(c2)-c2cccc4c2c-3cc2ccc3ccccc3c24)cc1] | 38.4 |
| Ph at 11 position | 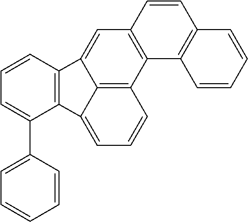 [SMILES: c1ccc(-c2cccc3c2-c2cccc4c2c-3cc2ccc3ccccc3c24)cc1] | 62.2 |
| Ph at 12 position | 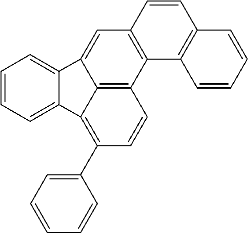 [SMILES: c1ccc(-c2ccc3c4c(cc5ccc6ccccc6c53)-c3ccccc3-c24)cc1] | 56.2 |
| Ph at 13 position | 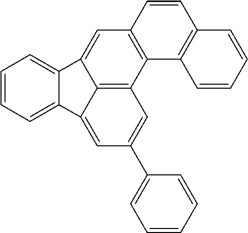 [SMILES: c1ccc(-c2cc3c4c(cc5ccc6ccccc6c5c4c2)-c2ccccc2-3)cc1] | 40.6 |
| Ph at 14 position | 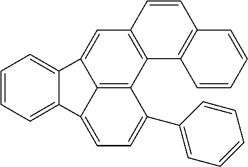 [SMILES: c1ccc(-c2ccc3c4c(cc5ccc6ccccc6c5c24)-c2ccccc2-3)cc1] | 48.5 |

According to Table 2, in a case where the naphtho[2,1-b]fluoranthene backbone binds a phenyl group at the 3 position, the resulting dihedral angle is the smallest. Although it is preferable that a dihedral angle in the compound backbone is larger, it is preferable that a dihedral angle between the main backbone and substituent is smaller. What is meant by the phrase “the dihedral angle is smaller” is that that stress between the main backbone and the benzene ring is small, and the binding distance is short with the binding energy being large. What is meant by the phrase “substituent of binding energy is large” is that bonds between the main backbone and the substituent are strong, and that it is difficult to cleave the substituent from the main backbone.

Namely, a compound of which dihedral angle is small is superior in stability.

From the above, the naphtho[2,1-b]fluoranthene compound according to aspects of the present invention is a high stability compound, since the compound has an aromatic hydrocarbon group at the 3 position as a substituent.

Herein, the aromatic hydrocarbon group substituted at the 3 position of the naphtho[2,1-b]fluoranthene backbone may be selected from an aromatic hydrocarbon group for which the S1 value is larger than the S1 energy of the naphtho[2,1-b]fluoranthene backbone. Specifically, an aromatic hydrocarbon group having 6 to 24 carbon atoms is preferable.

More specifically, the aromatic hydrocarbon group represented by Ar₁ and Ar₂ in the formula [1] may be a monovalent of bivalent aromatic hydrocarbon group selected from the group consisting of a benzene, biphenyl, terphenyl, naphthalene, fluorene, phenanthrene, chrysene, fluoranthene and triphenylene. The above 9 types of aromatic hydrocarbon group are due to the S1 energies thereof and in view of point (2).

The naphtho[2,1-b]fluoranthene compound may be substituted by an alkyl group having 1 to 4 carbon atoms, an alkoxy group having 1 or 2 carbon atoms, a halogen atom or cyano group at the 9 and the 10 position of the naphtho[2,1-b]fluoranthene backbone, as R₁ and R₂ in the formula [1]. These substituents are used for auxiliary modifications of the naphtho[2,1-b]fluoranthene backbone, main backbone, and the coordinating properties of compound. The alkyl group which has a high stability may be preferable in these substituents.

Similarly, the aromatic hydrocarbon group represented by Ar₁ and Ar₂ in the formula [1] of the naphtho[2,1-b]fluoranthene compound according to aspects of the present invention may be substituted by an alkyl group having 1 to 4 carbon atoms, an alkoxy group having 1 or 2 carbon atoms, a halogen atom or a cyano group, as a substituent. These substituents are used for auxiliary modifications to the naphtho[2,1-b]fluoranthene backbone, main backbone, and coordinating properties of compound. The alkyl group which has a high stability is preferable for these substituents.

In addition, in a case where the naphtho[2,1-b]fluoranthene compound according to aspects of the present invention is used for a component material of an organic light-emitting device, it is preferable to enhance the purity of the compound by performing a purification method by sublimation. This is because the purification method by sublimation exerts a large effect in enhancing the purity of an organic compound. In the purification method by sublimation, the higher the molecular weight of the compound is, the higher the temperature that is needed. Also, in the case where a high temperature being used, it is easier to decompose the compound. Therefore, molecular weight of an organic compound used for a component material of an organic light-emitting device is preferably 1000 or less, so as to enable the purification method by sublimation to be performed without excessive heat. The molecular weight of the organic compound is more preferably 800 or less, and n in the formula [1] is selected from 0 or 1.

### Concrete structure of the naphtho[2,1-b]fluoranthene

A concrete structure of the naphtho[2,1-b]fluoranthene according to aspects of the present invention is exemplified below. However, aspects of the present invention are not limited to these compounds.

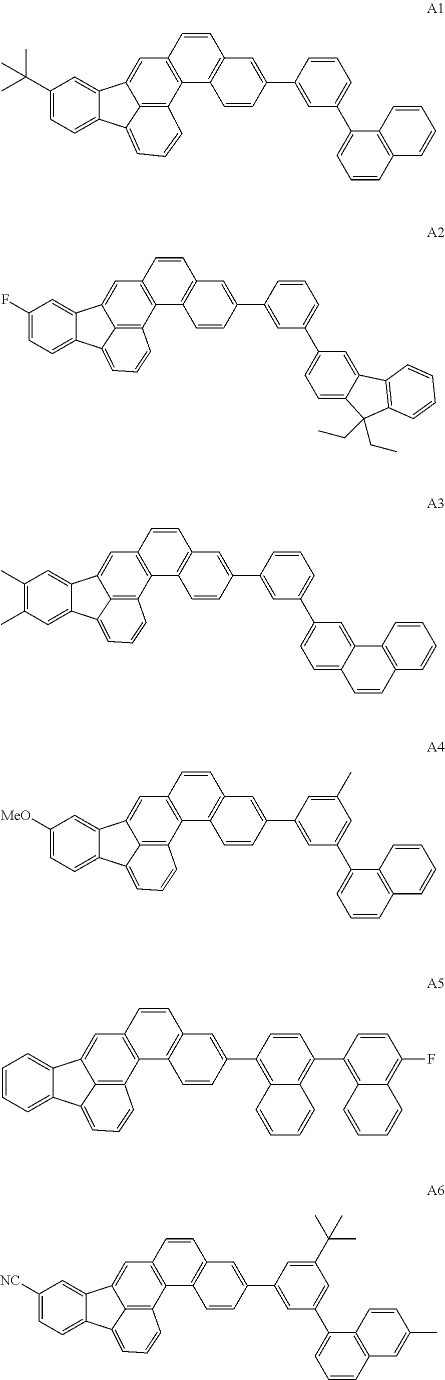

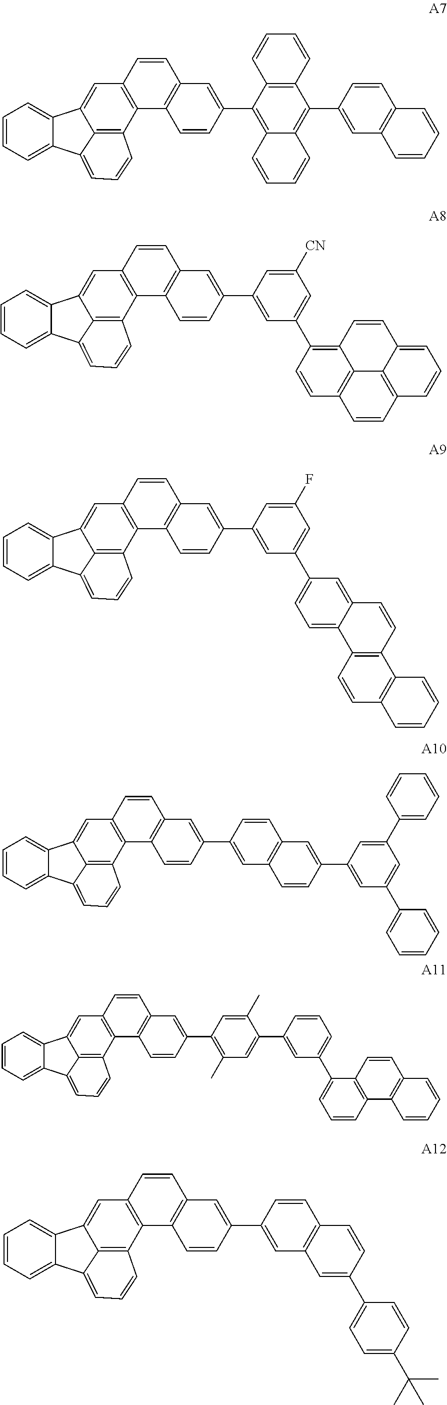

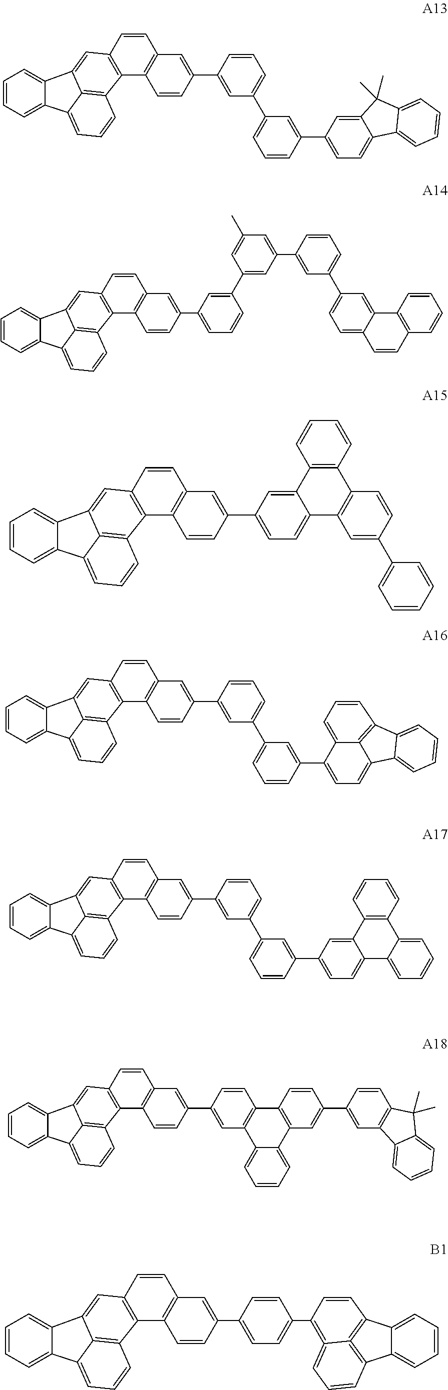

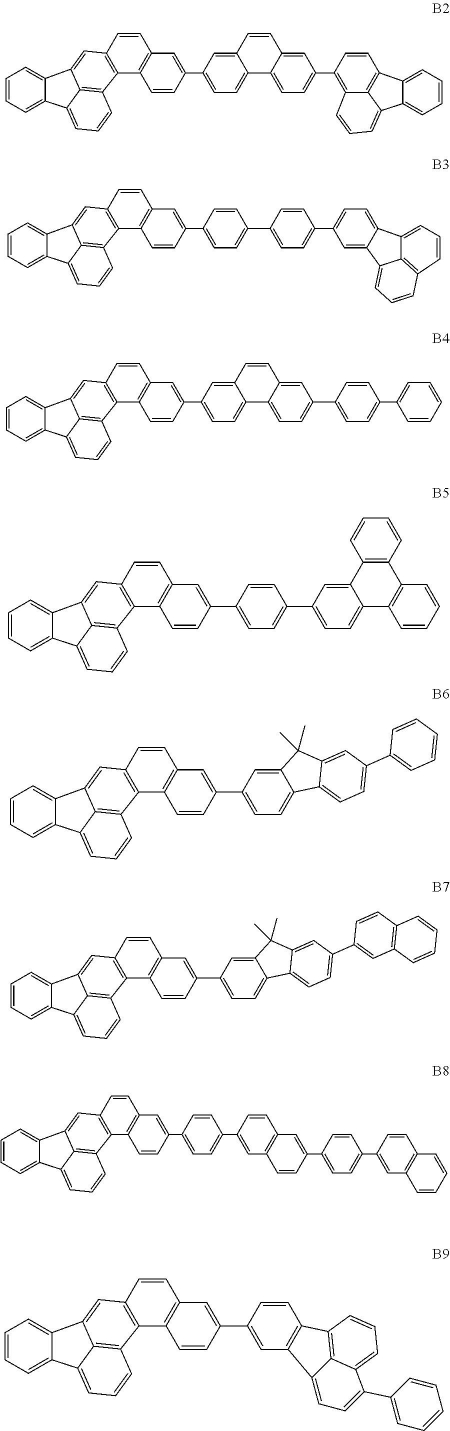

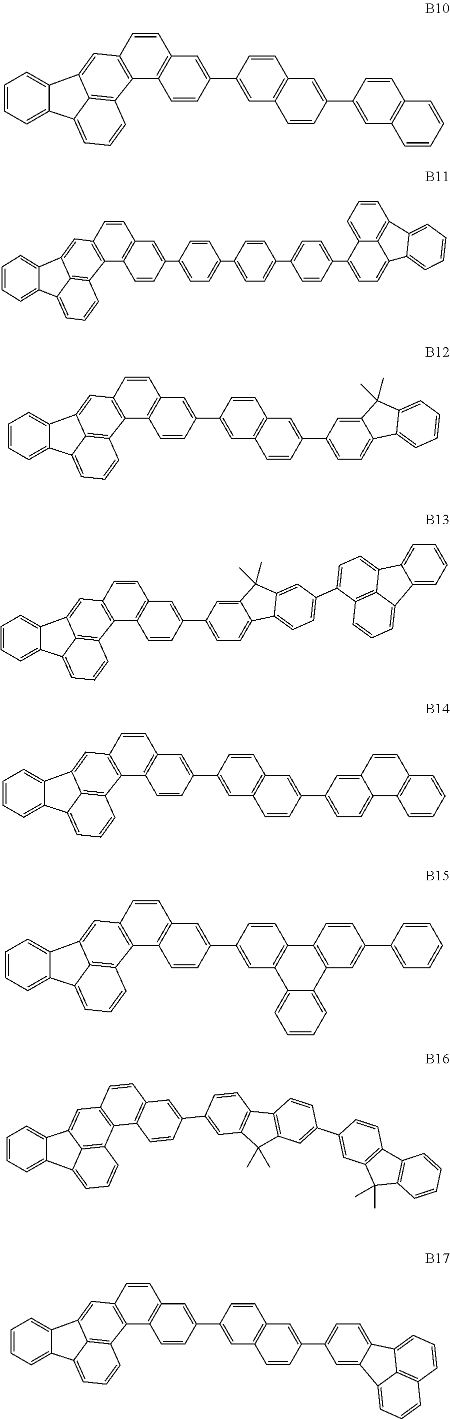

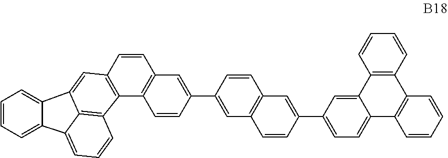 [SMILES: c1ccc2c(c1)-c1cccc3c1c-2cc1ccc2cc(-c4ccc5cc(-c6ccc7c8ccccc8c8ccccc8c7c6)ccc5c4)ccc2c13]

Among the exemplified compounds, the bonding of Ar₁ and Ar₂ in the formula [1] of the compounds shown in the A group provides a compound that is nonlinear. The compound shown in A group is capable of forming a film of good properties and an amorphous film of high stability.

On the other hand, the bonding of Ar₁ and Ar₂ in the formula [1] of the compounds shown in the B group forms a compound that is more linear and extended. The compounds shown in B group have high electron mobility.

### Synthesis method of the naphtho[2,1-b]fluoranthene compound

Next, a synthesis method of the naphtho[2,1-b]fluoranthene compound according to aspects of the present invention is explained below.

The naphtho[2,1-b]fluoranthene compound according to aspects of the present invention embodiment can be synthesized, for example, according to a synthetic scheme shown in the following general formula [6].

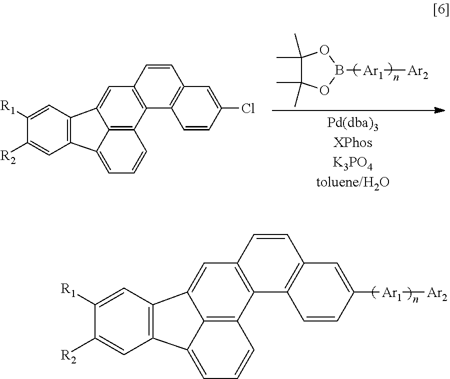

In the formula [6], each of R₁, R₂, Ar₁, Ar₂ and n is the same as those in the general formula [1].

Specifically, the compound can be synthesized using following compounds (A) and (B) by cross-coupling reaction with Pd catalyst.

(A) a compound substituted by chlorine atom at the 3 position of the naphtho[2,1-b]fluoranthene compound (B) Pinacol borane ester compound to which are bounded (Ar₁)ₙ—Ar₂ as substituents. Also, the following compounds (A′) and (B′) may be used instead of the compounds (A) and (B).

(A′) a compound substituted by pinacol borane ester at the 3 position

(B′) a compound represented by Hal-(Ar₁)ₙ—Ar₂ (Hal denotes a halogen atom)

Herein, the compound (A) can be synthesized, for example, according to a synthetic scheme shown in following general formula [7].

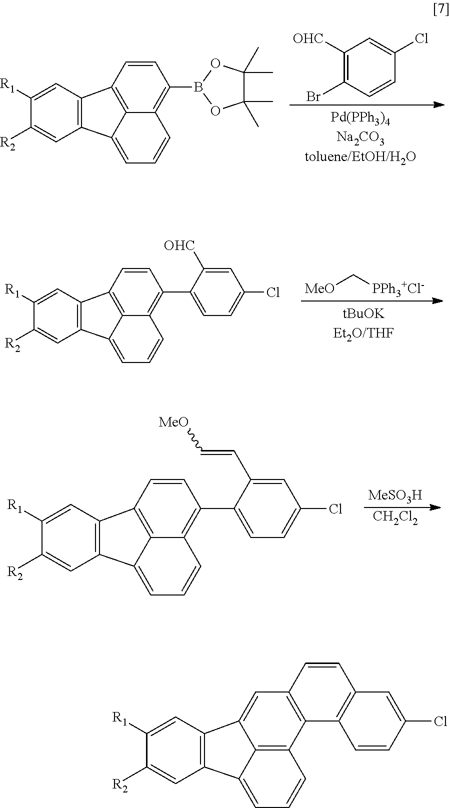

In formula [7], R₁ and R₂ are the same as R₁ and R₂ in the formula [1], respectively.

Specifically, the naphtho[2,1-b]fluoranthene compound according to aspects of the present invention may be synthesized by performing following reactions (1) to (3) in this order with pinacol borane ester body of the naphtho[2,1-b]fluoranthene compound having intended substituents and 2-bromo-5-chloro-benzaldehyde. Namely, the naphtho[2,1-b]fluoranthene compound according to aspects of the present invention may be synthesized by properly changing substituents on the basis of these synthetic examples.

(1) cross-coupling reaction with Pd catalyst
(2) Wittig reaction
(3) Acid cyclization reaction

### Organic Light-Emitting Device

Next, the organic light emitting element according to an embodiment of the present invention will be described.

The organic light-emitting device according to this embodiment contains at least an anode and a cathode which are a pair of electrodes facing each other, and an organic compound layer arranged between these electrodes. The organic light-emitting layer contains at least a light-emitting layer. In the organic light emitting device of the present embodiment, the organic compound layer may contain the naphtho[2,1-b]fluoranthene compound according to aspects of the present invention. In addition, it is preferable that a hole blocking layer thereof contains the naphtho[2,1-b]fluoranthene compound according to aspects of the present invention. The hole blocking layer is arranged at the cathode side of the light-emitting layer, between the light-emitting layer and the cathode.

As the element constitution of the organic light-emitting element according to aspects of the present invention, a multilayered device configuration in which organic compound layers given below are sequentially laminated on a substrate is mentioned.

(1) anode/light-emitting layer/cathode
(2) anode/hole transporting layer/light-emitting layer/electron transporting layer/cathode
(3) anode/hole transporting layer/light-emitting layer/electron transporting layer/electron injection layer/cathode
(4) anode/hole injection layer/hole transporting layer/light-emitting layer/electron transporting layer/cathode
(5) anode/hole injection layer/hole transporting layer/light-emitting layer/electron transporting layer/electron injection layer/cathode
(6) anode/hole transporting layer/electron blocking layer/light-emitting layer/hole blocking layer/electron transporting layer/electron injection layer/cathode

It should be noted that the above element constitutions are merely provided as examples, and the constitution of the organic light emitting elements of the embodiment is not limited thereto.

Furthermore, different layer configurations can be provided such as, for example, an insulating layer that is provided at an interface between an electrode and an organic compound layer, an adhesive layer or an interference layer, an electron transporting layer or a hole transporting layer that is contains a plurality of layers with different ionization potentials, a light-emitting layer that contains a plurality of layers with different light emitting materials, and the like.

According to one embodiment, among the above-mentioned element configurations, the configuration (6) containing both an electron blocking layer and a hole blocking layer is preferably used. In the configuration (6), both carriers, holes and electrons, can be blocked in a light-emitting layer, and thus a light-emitting element with high light emission efficiency without carrier leakage can be obtained.

In one embodiment, as a component material for each layer of an organic light-emitting device, a known material other than the naphtho[2,1-b]fluoranthene compound may be used. An organic compound or an inorganic compound may be used as a material for each of the layers. And a material for each of the layers may be a low molecular weight compound or a polymer.

### About the Anode

The constituent materials of the anode are preferably those which have a work function as high as possible, specifically 4.5 eV or more and 5.5 eV or less. For example, simple metals such as aurum, platinum, silver, copper, nickel, palladium, cobalt, selenium, vanadium and tungsten, or alloys in which these are combined, metal oxides such as tin oxide, zinc oxide, indium oxide, indium tin oxide (ITO) and indium zinc oxide can be used. In addition, conductive polymers such as polyaniline, polypyrrole and polythiophene can be also used.

These electrode substances may be used alone or two or more of the electrode substances may be used in combination. In addition, the anode may be constituted of a layer or may be constituted of a plurality of layers. About the hole transporting layer and the hole injection layer

As the hole injection/transporting materials, a material with high hole mobility is preferred so that holes can be easily injected from an anode and injected holes can be transported to a light emitting layer. In addition, in order to prevent film deterioration such as crystallization in an organic light emitting element, a material with high glass transition temperature is preferred. As the low molecular and high molecular materials with hole injection/transport properties, triarylamine derivatives, arylcarbazole derivatives, phenylenediamine derivatives, stilbene derivatives, phthalocyanine derivatives, porphyrin derivatives, poly(vinylcarbazole), poly(thiophene), other conductive polymers are mentioned. Further, the above-mentioned hole injection/transport materials are also suitably used for an electron blocking layer. Specific examples of compounds used as a hole injection/transporting material are shown below. The compounds to be used are, however, not limited thereto.

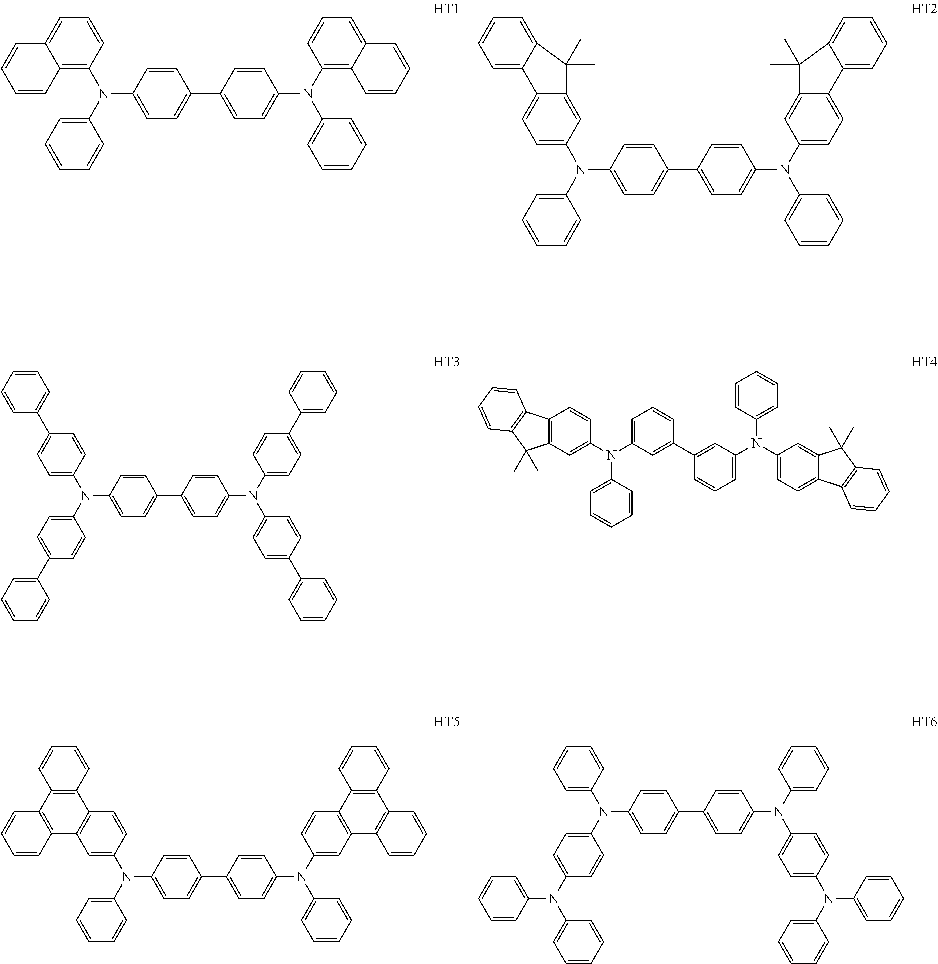

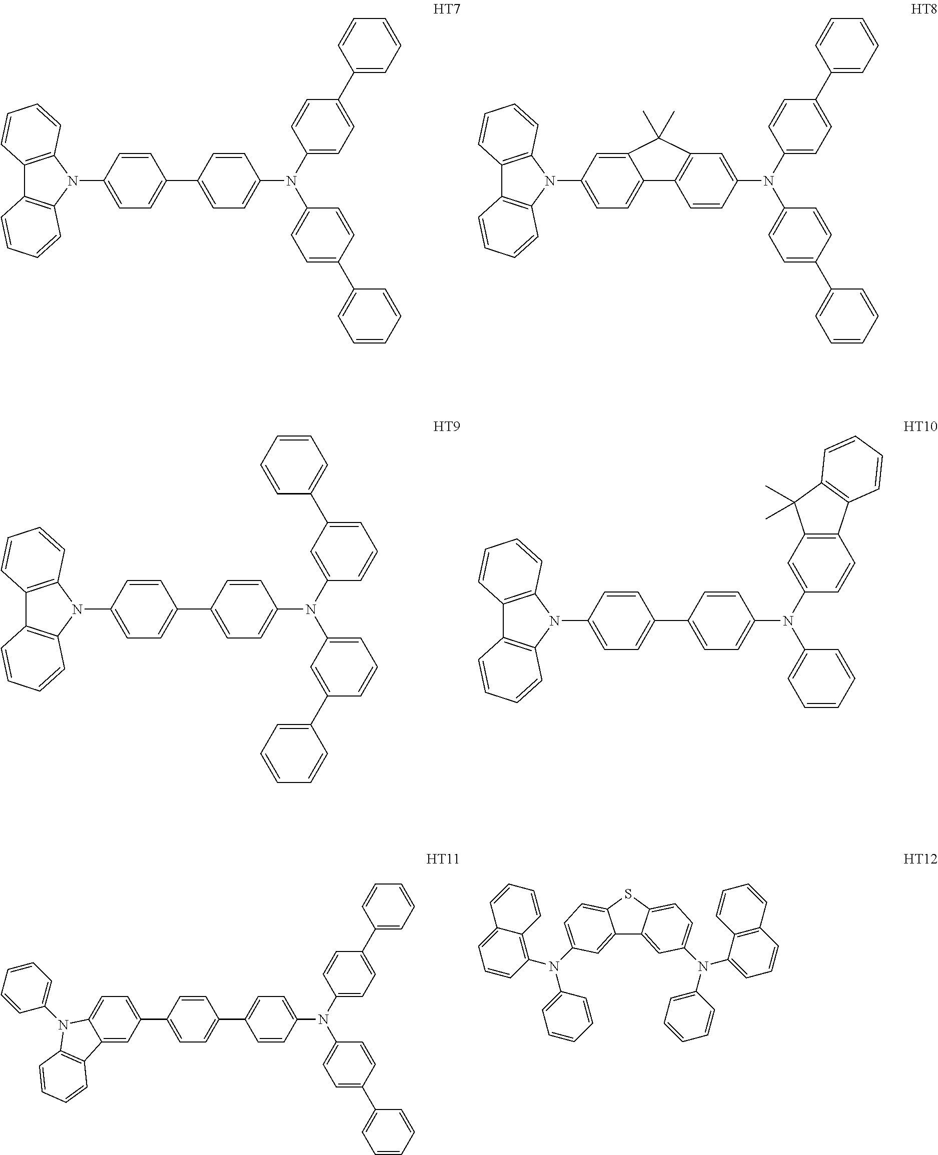

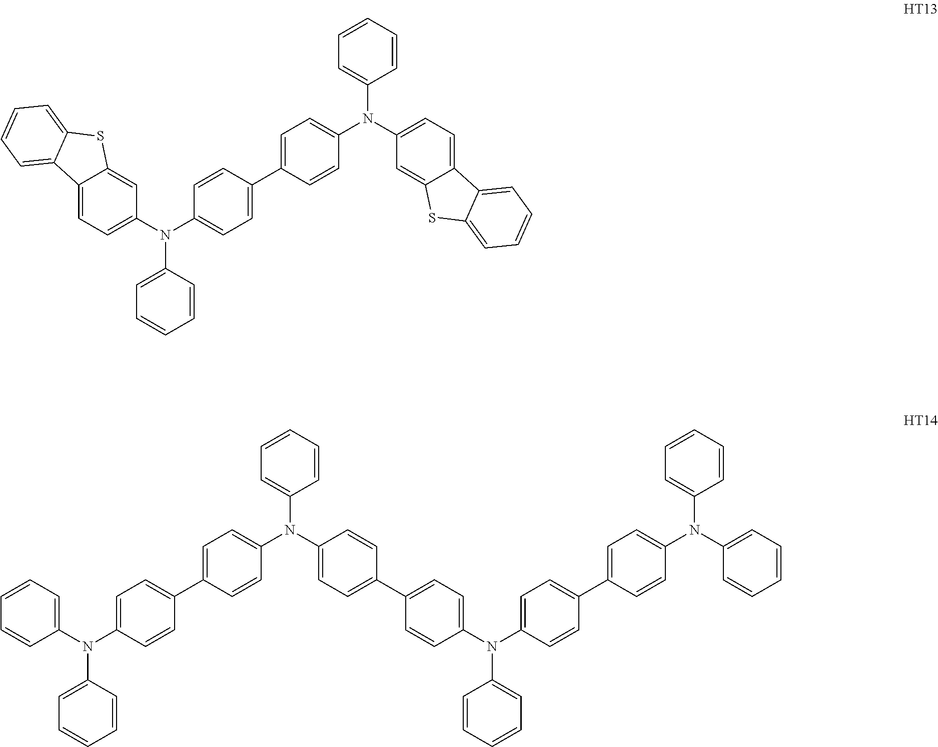 [SMILES: c1ccc(N(c2ccc(-c3ccc(N(c4ccccc4)c4ccc5c(c4)sc4ccccc45)cc3)cc2)c2ccc3c(c2)sc2ccccc23)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

### About the Light-Emitting Layer

The light-emitting layer of the organic light-emitting device according to the present embodiment may be a light-emitting layer which contains only a light-emitting material, and is preferably contains the light-emitting material and another type of material. The light-emitting material denotes a guest material of a light-emitting layer or a dopant of a light-emitting layer. Namely, the light-emitting layer preferably contains a host and a guest.

The host of the light emitting layer is a material that does not aggregate around a guest, such as, a material existing as matrix. And the host is a material for transporting a carrier to a guest of a light emitting layer or donating excitation energy to the guest.

The guest of the light-emitting layer is a compound responsible for main light emission in a light emitting layer. The guest is also referred to as a light-emitting dopant.

The concentration of the host in the light-emitting layer is 50 wt % or more and 99.9 wt % or less relative to the total weight of the light emitting layer.

The concentration of the guest in the light-emitting layer is 0.01 wt % or more and 50.0 wt % or less and preferably 0.1 wt % or more and 20.0 wt % or less relative to the total weight of the light-emitting layer. In order to prevent concentration quenching, the concentration of the guest material is especially preferably 0.01 wt % or more and 10 wt % or less.

The concentration of the material in the light-emitting layer may be measured by instrumental analysis. Specifically, mass spectrometry, liquid chromatography or gas chromatography is exemplified.

In the layer including a host, a guest may be entirely uniformly contained, or may be contained with a concentration gradient, for example, changing the concentration of guest with closing the cathode from the anode. The guest may be contained in a specific region to provide a host layer region without the guest material.

In addition, the light-emitting layer may contain an emitting assist material or a charge injection material other than the host and the guest of the light-emitting layer. The concentration of the assist material is less than that of the host and the charge injection material is responsible for carrier injection from a carrier transporting layer e.g. layer adjacent to the light-emitting layer, to the-light emitting layer.

As the light-emitting materials, fused ring compounds such as fluoranthene derivatives, fluorene derivatives, naphthalene derivatives, pyrene derivatives, perylene derivatives, tetracene derivatives, anthracene derivatives, rubrene, organic compound such as quinacridone derivatives, coumarin derivatives, stilbene derivatives, organic aluminum complexes such as tris(8-quinolinolate)aluminum, iridium complexes, platinum complexes, rhenium complexes, copper complexes, europium complexes, ruthenium complexes, and polymer derivatives such as poly(phenylenevinylene) derivatives, poly(fluorene) derivatives and poly(phenylene) derivatives are exemplified.

The fluoranthene derivative denotes a compound in which a fluoranthene backbone is provided with a substituent and a compound in which a fluoranthene backbone is provided with a condensed ring. It is similar about the other derivatives.

Specific examples of compounds used as light emitting materials will be now mentioned, but certainly not limited thereto.

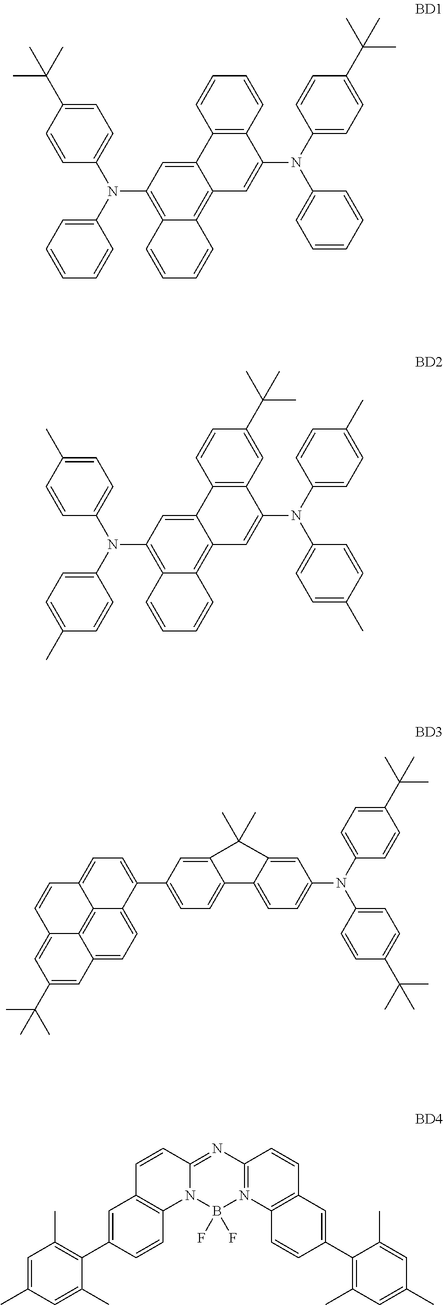

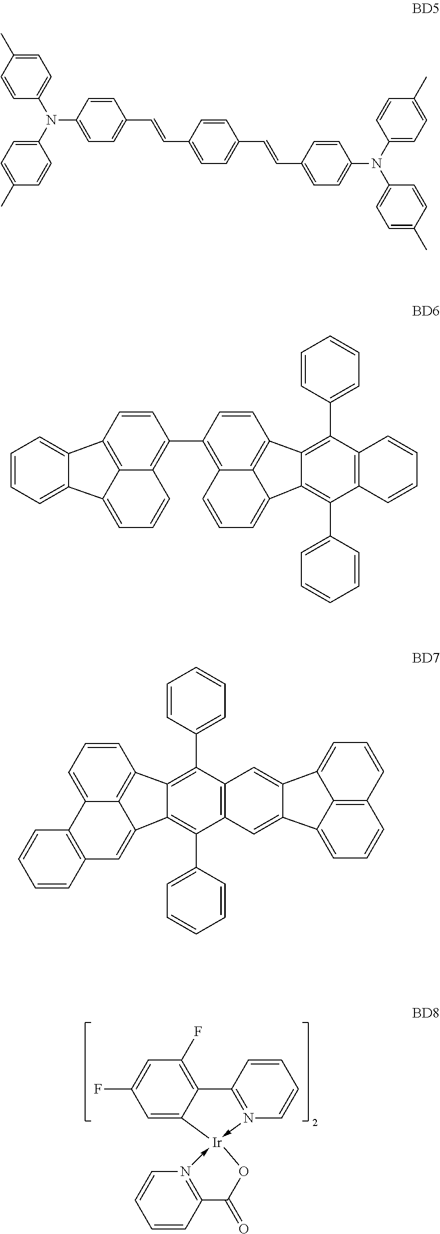

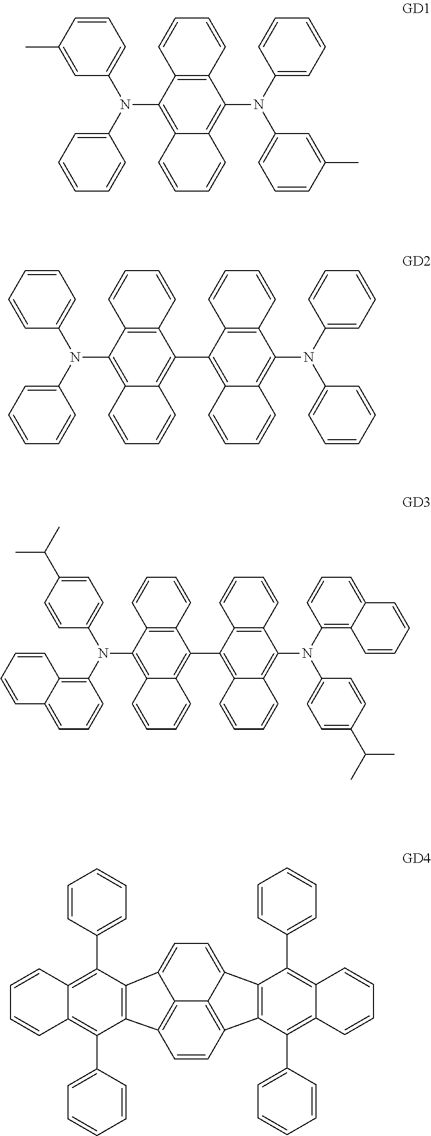

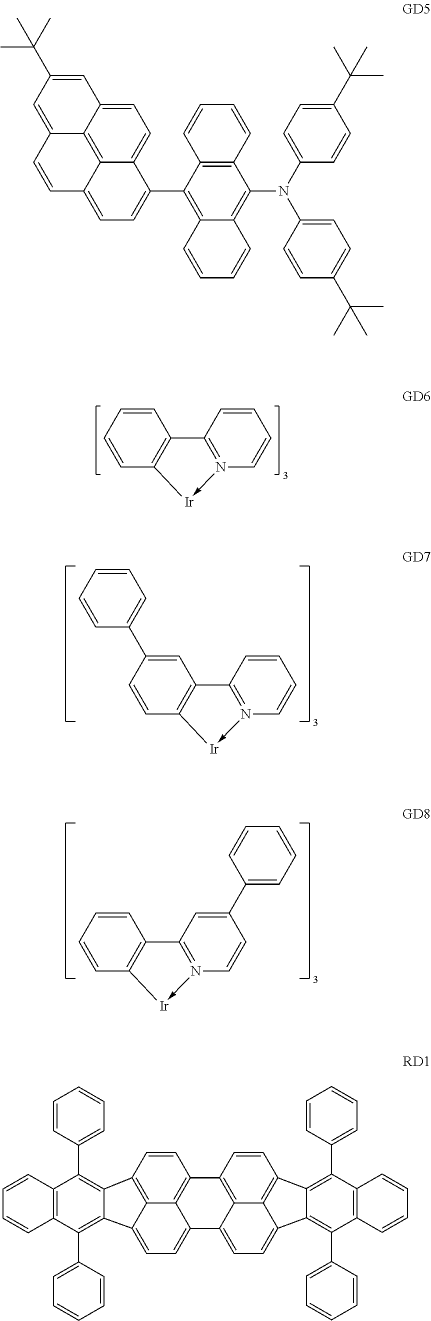

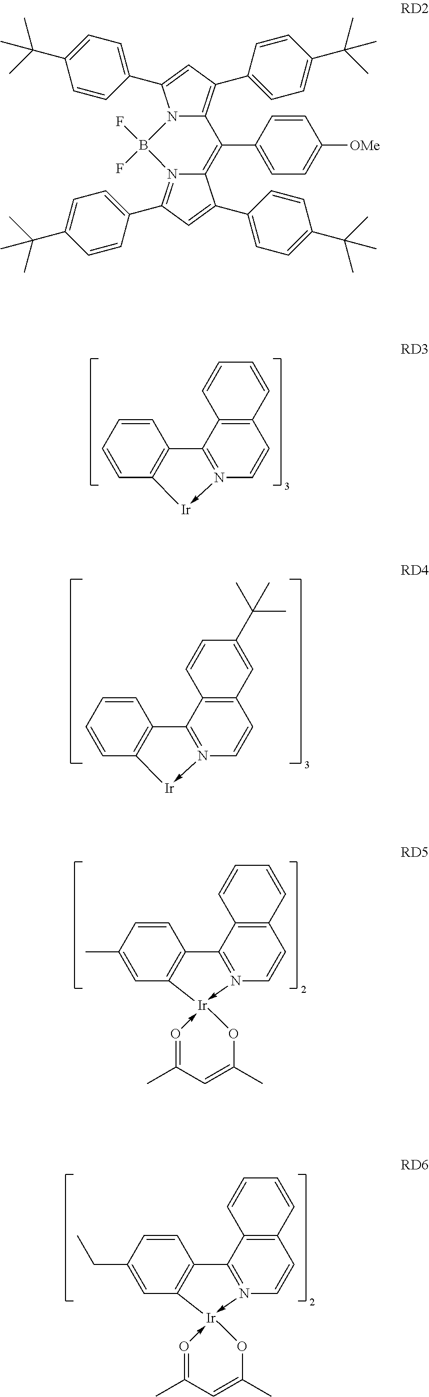

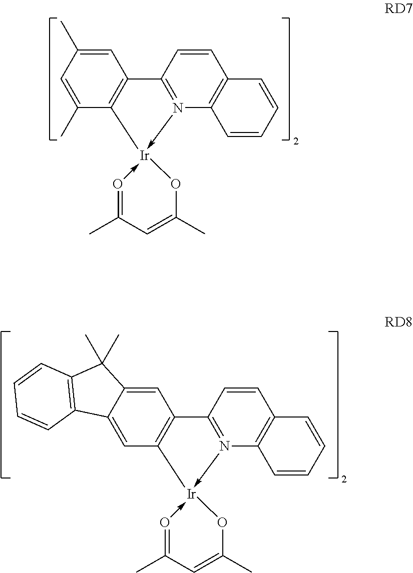

As the light-emitting layer hosts or the emitting assist materials contained in a light-emitting layer, in addition to aromatic hydrocarbon compounds or derivatives thereof, carbazole derivatives, dibenzofuran derivatives, dibenzothiophene derivatives, organic aluminum complexes such as tris(8-quinolinolate)aluminum, organic beryllium complexes and the like are exemplified.

Specific examples of compounds used as the light-emitting layer hosts or the emitting assist materials contained in a light-emitting layer will be now exemplified, but are certainly not limited thereto.

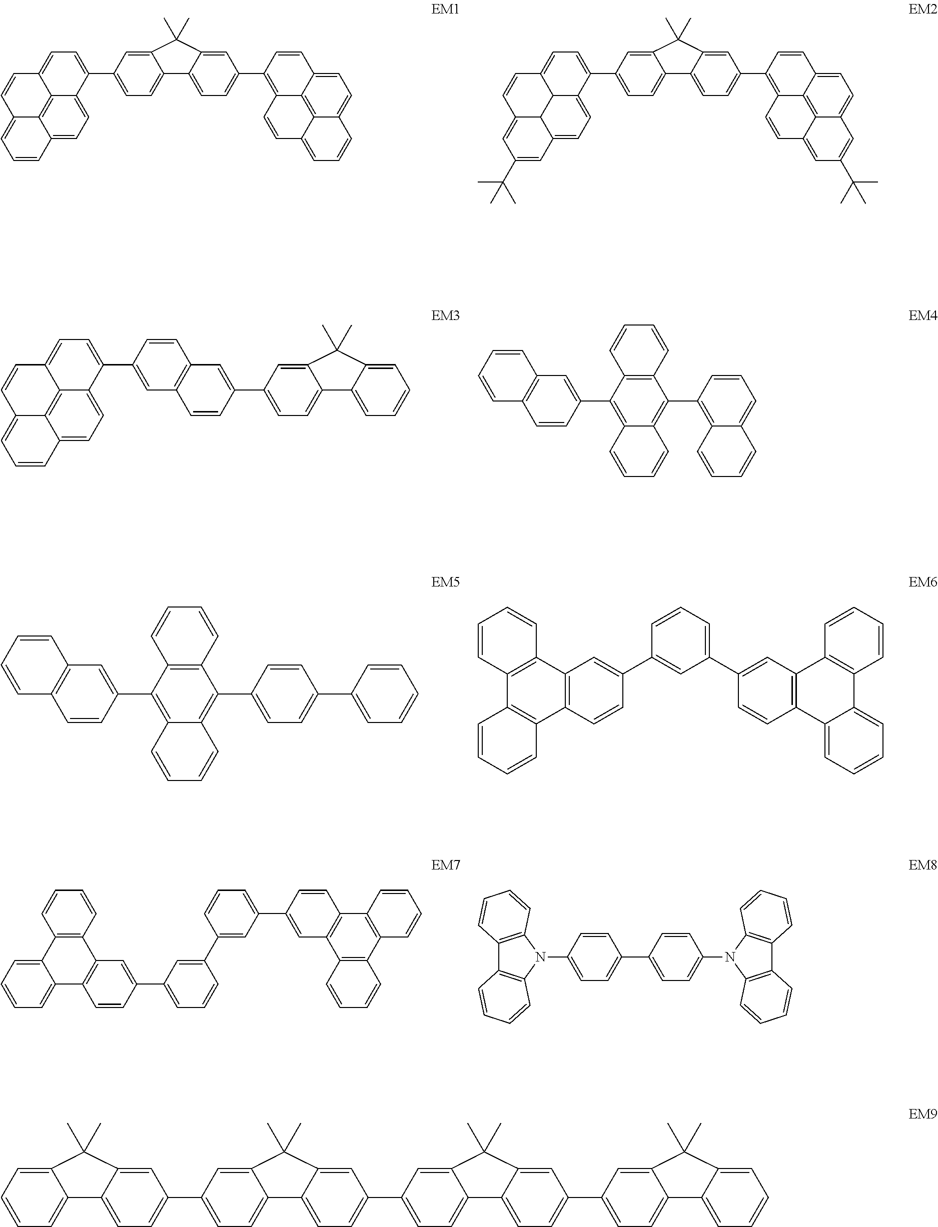

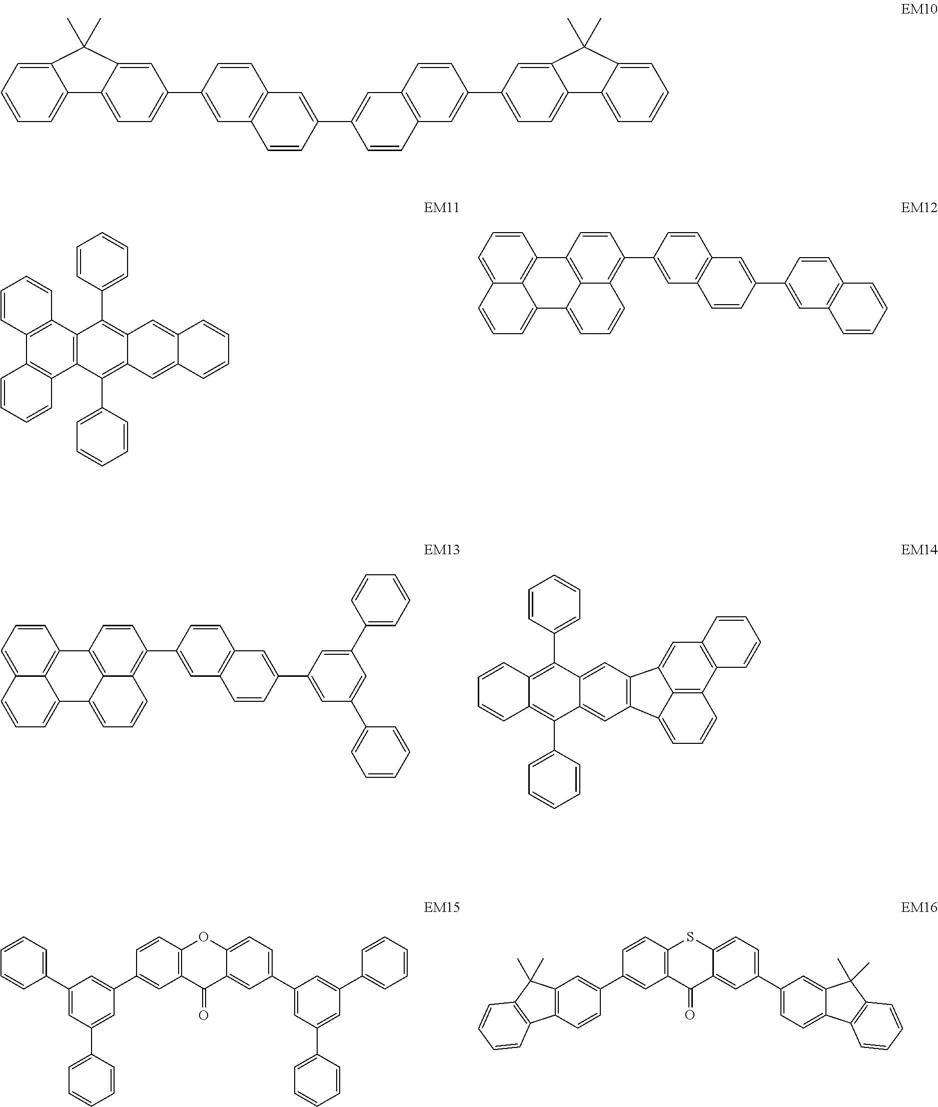

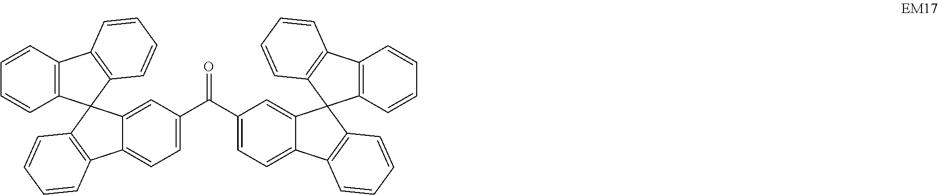 [SMILES: O=C(c1ccc2c(c1)C1(c3ccccc3-c3ccccc31)c1ccccc1-2)c1ccc2c(c1)C1(c3ccccc3-c3ccccc31)c1ccccc1-2]

In the organic light-emitting device according to the present embodiment, an organic compound layer contains a light-emitting unit, and this light-emitting unit can contain a plurality of light-emitting materials. Any two of the plurality of light-emitting materials may emit light of a different wavelength than the other, and a device containing these materials may be an device emitting a white color. However the emission color of the device is not limited.

In addition, the organic light-emitting device according to the present embodiment may contain a plurality of light emitting layers. Any two of the plurality of light emitting layers may emit light of a wavelength different than the other. In a case where an organic light-emitting device contains a plurality of light-emitting layers, the device may emit any color light, and preferably emits a white color. In a case where an organic light-emitting device contains a plurality of light-emitting layers, the plurality of light-emitting layers may be arranged tandem in the direction from the cathode to the anode, and may be arranged side by side in parallel.

The phrase “arrange side by side in parallel” indicates that the plurality of light emitting layers contact organic compound layers adjacent to the light-emitting layers. The layer adjacent to the light-emitting layer is a carrier injection layer and so on, and not a light-emitting layer.

### About the Electron Transporting Layer and the Hole Blocking Layer

A material for the electron transporting layer and the hole blocking layer is, other than the naphtho[2,1-b]fluoranthene compound according to the embodiment, a compound selected in view of the electron transport property, electron injection property from the cathode or electron injection layer, and hole blocking property. Specifically, oxadiazole derivatives, oxazole derivatives, pyrazine derivatives, triazole derivatives, triazine derivatives, quinoline derivatives, quinoxaline derivatives, phenanthroline derivatives, organic aluminum complexes, fused ring compounds (e.g. fluorene derivatives, naphthalene derivatives, chrysene derivatives, anthracene derivatives etc.) are mentioned. Further, the above-mentioned electron transporting materials are also suitably used for a hole blocking layer. Specific examples of compounds used as the electron transporting material and/or the hole blocking material will be now exemplified, but certainly not limited thereto.

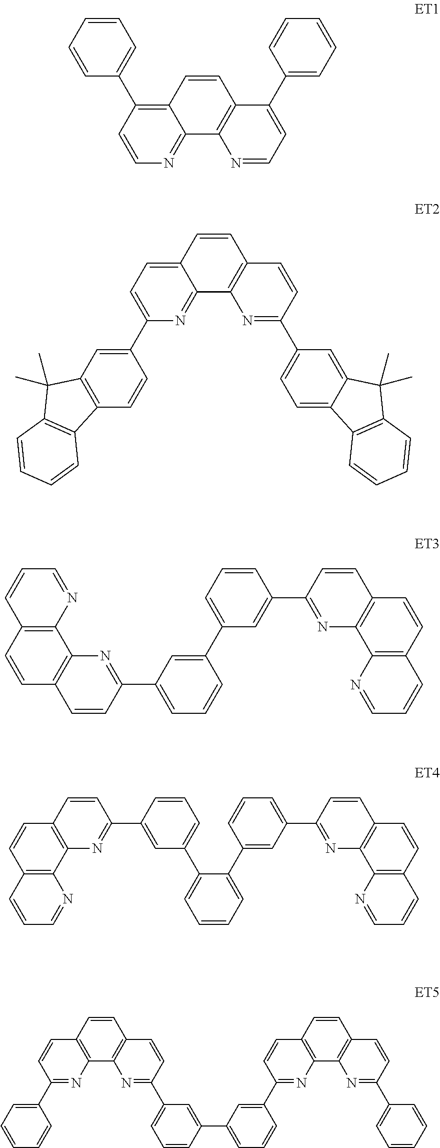

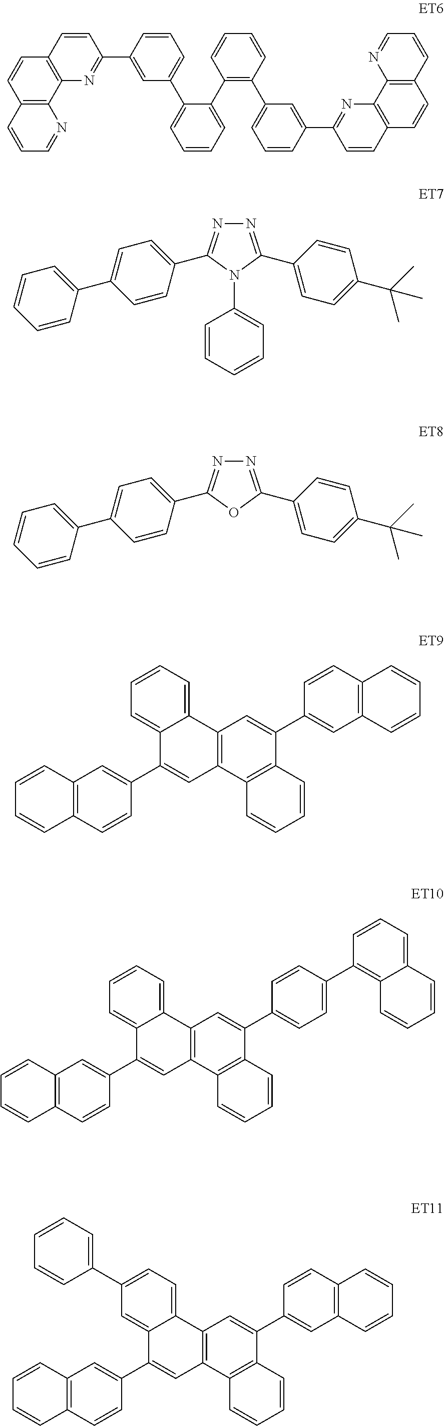

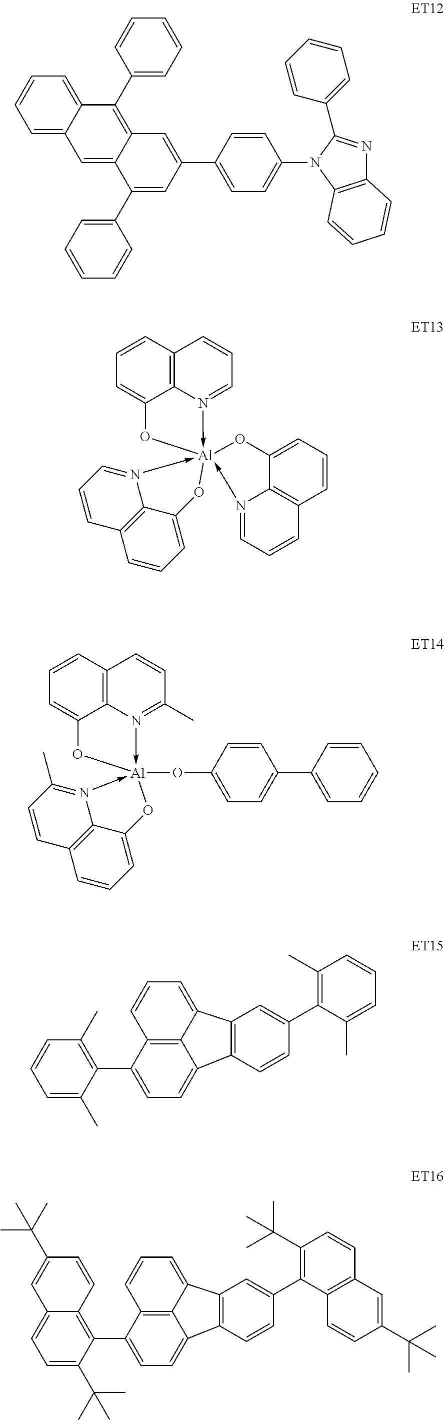

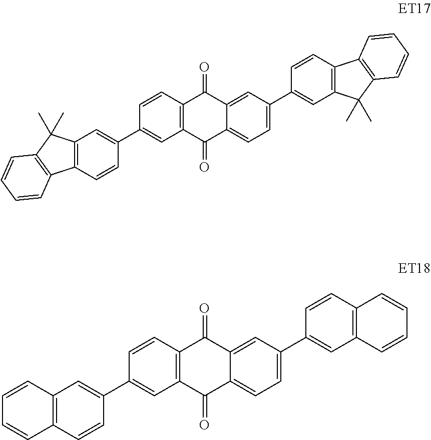 [SMILES: CC1(C)c2ccccc2-c2ccc(-c3ccc4c(c3)C(=O)c3ccc(-c5ccc6c(c5)C(C)(C)c5ccccc5-6)cc3C4=O)cc21 ; O=C1c2ccc(-c3ccc4ccccc4c3)cc2C(=O)c2ccc(-c3ccc4ccccc4c3)cc21]

The electron transporting material and the hole blocking material exemplified above may be contained in an organic compound layer arranged between an electron injection layer and a light emitting layer, such as an electron transporting layer or a hole blocking layer.

### About the Electron Injection Layer

The electron injection layer is a layer responsible for facilitating electron injection from the cathode, arranged between the electron transporting layer or the hole blocking layer and the cathode and contains reducible dopant as an electron injection material. The reducible dopant is normally used with a host of the electron injection layer, but it may be used independently.

In a case where the electron injection layer contains a host for electron injection layer, the concentration of the reducible dopant is 0.1 wt % or more and 80.0 wt % or less, preferably 1.0 wt % or more and 50.0 wt % or less, more preferably 5.0 wt % or more and 30.0 wt % or less on the basis of the total weight of the electron injection layer. Namely, the concentration of the other type of compound is preferably 20.0 wt % or more and 99.9 wt % or less.

As the reducible dopant contained in the electron injection layer, an alkali metal, an alkaline-earth metal, rare earth metal, the oxide of the alkali metal, the halogenide of the alkali metal, the carbonate of the alkali metal, an alkali metal complex, the oxide of the alkaline-earth metal, the halogenide of the alkaline-earth metal, alkali metal earths complex, the oxide of the rare earth metal, the halogenide of the rare earth metal, a rare earth metal complex and so on are exemplified.

Materials that are the same as those specifically exemplified as the compounds of the electron transporting material and the hole blocking material can be used for the host of the electron injection layer.

### About the Cathode

The constituent materials of the cathode are preferably metals which have a low work function. Specifically, the work function is 2.0 eV or more and 5.0 eV or less. It is noted that the metals include the oxide thereof in the embodiment.

Examples thereof include alkali metals such as lithium, alkaline earth metals such as calcium, and simple metals such as aluminum, titanium, manganese, silver, lead and chromium. Alternatively, alloys in which these simple metals are combined can be also used. For example, magnesium-silver, aluminum-lithium, aluminum-magnesium and the like can be used. Metal oxides such as indium tin oxide (ITO) can be utilized. These electrode substances may be used alone or two or more of the electrode substances may be used in combination. In addition, the cathode may be constituted of a layer or may be constituted of multi layers.

### Forming the Organic Compound Layer

Organic compound layers (a hole injection layer, a hole transporting layer, an electron blocking layer, a light-emitting layer, a hole blocking layer, an electron transporting layer, an electron injection layer etc.) constituted by the organic light emitting element according to aspects of the present invention are formed by the methods given below.

For the organic compound layers constituted by the organic light emitting element according to aspects of the present invention, dry processes such as a vacuum evaporation method, an ionized evaporation method, sputtering and plasma can be used. In addition, wet processes, that is, after dissolution in a proper solvent, a layer is formed by a known coating method (e.g. spin coating, dipping, a casting method, a LB method, an ink-jet method etc.), can be also used in place of dry processes.

In a case where a layer is formed by a vacuum evaporation method, a solution coating method or the like, crystallization and the like are less likely to occur and temporal stability is excellent. In addition, when a film is formed by a coating method, a film can be also formed by combining a proper binder resin therewith.

As the above-mentioned binder resins, polyvinyl carbazole resin, polycarbonate resin, polyester resin, ABS resin, acrylic resin, polyimide resin, phenol resin, epoxy resin, silicon resin, urea resin and the like are mentioned, but not limited thereto.

In addition, these binder resins may be used alone as a homopolymer or a copolymer or two or more of the binder resins may be used in combination. Further, known additives such as a plasticizer, an antioxidant and a UV absorber may be combined as needed.

### Another Embodiment of Organic Light-Emitting Device

The organic light-emitting device according to aspects of the present invention may provide a configuration for producing light from at least one of pair of electrodes.

The organic light-emitting device is disposed on a substrate such as a glass substrate or a silicone substrate. The organic light-emitting device may produce light from the substrate side, i.e. a bottom emission system, and from the opposite side of the substrate, i.e. a top emission system and from the both sides of the substrate.

In case of the bottom emission system, the substrate is a light transmission substrate. In case of the top emission system, the electrode arranged on the side far from the substrate is a thin film electrode and is a light transmission electrode. In this case, the substrate may be a transmissive substrate or a non-transmissive substrate. As the non-transmissive substrate, exemplified is a silicon substrate.

### Uses of the Organic Light-Emitting Device

The organic light-emitting device according to aspects of the present invention can be used as a constituent member for a display device and a lighting device. Further, there are uses such as an exposure light source of an electrophotographic image forming device in which a latent image is formed on a photosensitive member, a backlight of a liquid crystal display device, a color filterless white light source and a light-emitting device having a color filter and a white light source and the like. The color filter is a filter into which at least any of, for example, three colors, red, green and blue, penetrates. A light-emitting device combining a filter to adjust the chromaticity of a white color and a white light source can be used.

The display apparatus contains a plurality of pixels in its display unit. At least one of the plurality of pixels comprises the organic light-emitting device of the present embodiment and an active element connected to the organic light emitting element. A switching element or an amplifying element can be exemplified as an active element. In this organic light-emitting element, the anode or the cathode thereof is electrically connected to the drain electrode or the source electrode of a transistor. The active element controls the pixels to emit light or not emit light.

The display unit has a detached space between one pixel and another pixel, each pixel being among the plurality of pixels. The organic compound layer may be disposed across the pixel and the detached space in succession.

The display apparatus can be used as an image display apparatus of PC and the like.

The transistor may have a semiconductor material such as silicon, may have an organic semiconductor material made of organic compound, and may have an oxide semiconductor in active region thereof.

A TFT(Thin Film Transistor) is exemplified as the transistor.

The display apparatus comprises a plurality of pixels. The plurality of pixels may be separated by a detached space where element separating layers such as banks are arranged in the direction toward the aspect. The organic light-emitting device contains an electron injection layer. The electron injection layer may be disposed across an organic light-emitting device adjacent to the organic light-emitting device and the detached space in succession. In other words, the plurality of organic light-emitting devices have the electron injection layer in common. Specifically, the electron injection layer may be formed on the entirety of a display region in the display apparatus all at once by a vacuum evaporation method or the like.

The display apparatus may be an image processing apparatus which contains an input unit to input image information from an area CCD, a linear CCD, memory cards and the like, and may be an image display device to display the input image on a display unit.

The image processing apparatus may be, more specifically, an image capturing apparatus such as a digital camera or a video camera, or an ink jet printer. The display device according to aspects of the present invention may be disposed in a back operation unit or a view finder. The display device according to aspects of the present invention may also be disposed in an operation unit of the ink jet printer. When the organic light emitting element according to aspects of the present invention is used for a display unit, a touch panel function can be contained. Touch panel function systems may be via a capacitive method, a resistive method or an infrared method.

The display device may be also used as a display unit of a multifunction printer.

In a case where the display apparatus is an image display apparatus used for a PC monitor, the organic light-emitting device included in a pixel or a sub pixel may emit light any colors of red, blue, green and may emit a color other than the three primary colors such as yellow.

Also, the organic light-emitting device according to aspects of the present invention may comprise a plurality of types of emission materials which emit colors that are different from each other, and thus may emit a white color. For example, each of the organic light-emitting devices may further comprise a color filter of red, green and blue and then the display apparatus which is able to display all the colors may comprise the organic light-emitting device.

The lighting device is a device used for illuminating an interior of a room. In the present embodiment, the white color has, for example, a color temperature of 4200 K and the neutral white color has, for example, a color temperature of 5000 K.

The lighting device comprises the organic light-emitting device according aspects of the present invention and an AC/DC converter connected to the organic light-emitting device. The lighting device may further contain a color filter.

In addition, the lighting device according to aspects of the present invention may contain a heat release unit that releases heat from a light-emitting unit and a circuit to the outside of the device. As the heat release unit, a radiator plate constituted of a metal with high thermal conductivity, a liquid silicon and the like are mentioned. As metals with high thermal conductivity, for example, metals having aluminum are mentioned. When liquid silicon is used in the heat release unit, heat can be released by convection of liquid silicon.

The image forming device according to aspects of the present invention is an image forming device comprising a photo conductor, an electrification unit to electrify the surface of this photo conductor, an exposure unit to expose the photo conductor, and a developing unit to develop an electrostatic latent image formed on the surface of the photo conductor, and a means of exposure contains the organic light-emitting element according to aspects of the present invention.

The organic light-emitting device according to aspects of the present invention may be used as a constituent part of an exposing apparatus that exposes a photosensitive member. In the exposing apparatus, for example, the organic light-emitting devices according to aspects of the present invention may be arranged in a line. Specifically, the plurality of the light-emitting devices are arranged in a line in a longitudinal direction of the photosensitive member in the exposure apparatus. The line may be one line or a plurality of lines, preferably 4 lines or less.

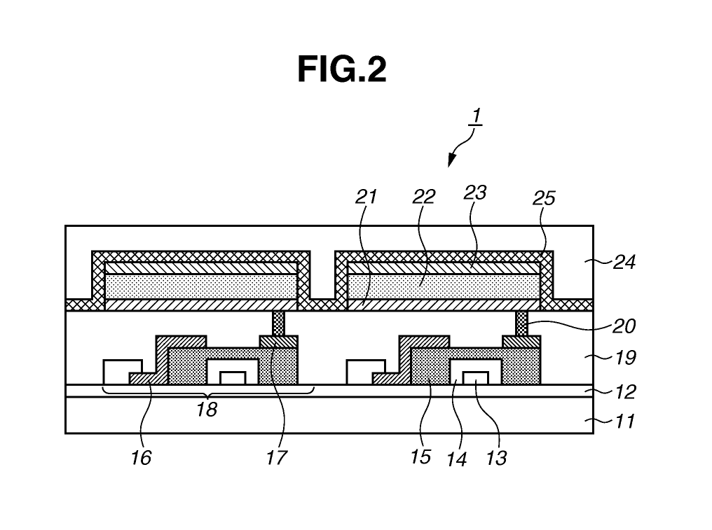

The image display device using the organic light emitting device of the present embodiment will be now described using FIG. 2. FIG. 2 is a sectional schematic view of an example for an image display apparatus. It is shown in FIG. 2 that two organic light-emitting devices and two TFT elements are each connected to a different one of the organic light-emitting devices.

In the image display device 1 in FIG. 2, a glass substrate 11 and a moisture proof film 12 to protect a TFT element or organic compound layers on the upper part thereof are provided. Further, a metallic gate electrode 13, a gate insulating film 14 and a semiconductor layer 15 are provided in the image display device 1.

A TFT element 18 contains the semiconductor layer 15, a drain electrode 16 and s source electrode 17. An insulating film 19 is provided on the upper part of the TFT element 18. An anode 21 and the source electrode 17 constituting the organic light-emitting element are connected to each other via contact holes 20.

That is, it is only necessary that either the anode or the cathode is electrically connected to either the source electrode or the drain electrode of the TFT element.

In the image display device 1 in FIG. 2, the organic compound layers are shown as a single layer, but an organic compound layer 22 may also be the multilayer form. A first protection layer 25 and a second protection layer 24 to inhibit the deterioration of the organic light-emitting element are provided on the top side of a cathode 23.

In a case where the image display device shown in FIG. 2 emits white light, a light-emitting layer included in the organic compound layer 22 may be one layer containing a red emission material, green emission material and blue emission material. And the organic light-emitting device may comprise multiple light emitting layers each containing a different layer from among a red emitting layer, green emitting layer and blue emitting layer. These light emitting layers may be arranged side by side in parallel. These light emitting materials may also be included in one light emitting layer and forming domains.

In the image display device 1 in FIG. 2, a transistor is used as a switching element, but in place of this, a MIM element may be used as a switching element.

The transistor used in the image display device 1 in FIG. 2 is not limited to a transistor using a single crystal silicon wafer and may be a thin film transistor having an active layer on the insulating surface of a substrate. As the active layer, single crystal silicon, amorphous silicon, non-single crystal silicon such as microcrystalline silicon, non-single crystal oxide semiconductors such as indium zinc oxide and indium gallium zinc oxide, a transparent oxide semiconductor are mentioned. It is noted that a thin film transistor is also referred to as a TFT element.

The transistor contained in the image display device 1 in FIG. 2 may be formed within a substrate such as a Si substrate. The formation within a substrate means that a transistor is produced by processing a substrate such as a Si substrate itself. That is, having a transistor within a substrate can be also considered that the substrate and the transistor are formed together.

Whether or not a transistor is provided within a substrate is selected depending on resolution. In the case of a 1 inch QVGA resolution, for example, it is preferred that an organic light-emitting element be provided within a Si substrate.

The organic light-emitting device according to aspects of the present invention may contain a switching element to control the light emission of the organic-light emitting element. The switching element connected to the organic light-emitting element may contain an oxide semiconductor in a channel unit thereof. The oxide semiconductor may be amorphous or a crystal or a state in which both exists.

The crystal may be a single crystal, a microcrystal, or a crystal in which a specific axis such as the C axis is oriented, or a mixture of at least two of them.

The organic light-emitting device containing such a switching element may be used as an image display device in which each organic light-emitting element is provided as a pixel, or may be used as a lighting device, and also may be used as an exposure light source to expose a photo conductor of an electrophotographic image forming device such as a laser beam printer or a copying machine.

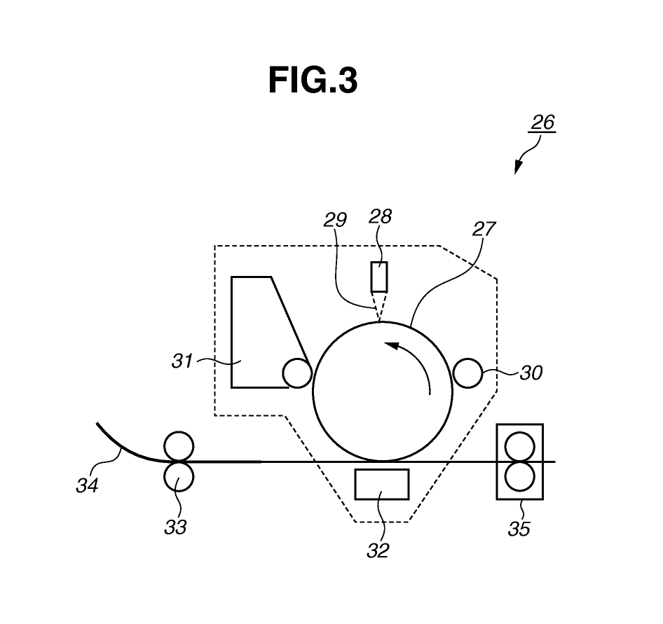

FIG. 3 is a schematic diagram of an image forming device 26 according to aspects of the present invention. The image forming device contains a photo conductor 27, an exposure light source 28, a developing machine 30, an electrification unit 31, a transcriber 32, a conveying roller 33 and a fuser 35.

Light 29 is emitted from the exposure light source 28 and an electrostatic latent image is formed on the surface of photo conductor 27. This exposure light source contains the organic light-emitting element according to aspects of the present invention. The developing machine 30 contains toner and the like. The electrification unit 31 electrifies the photo conductor 27. The transcriber 32 transfers a developed image onto a recording medium 34. The conveying roller 33 conveys the recording medium. The recording medium 34 is for example paper. The fuser 35 fixes the formed image on the recording medium.

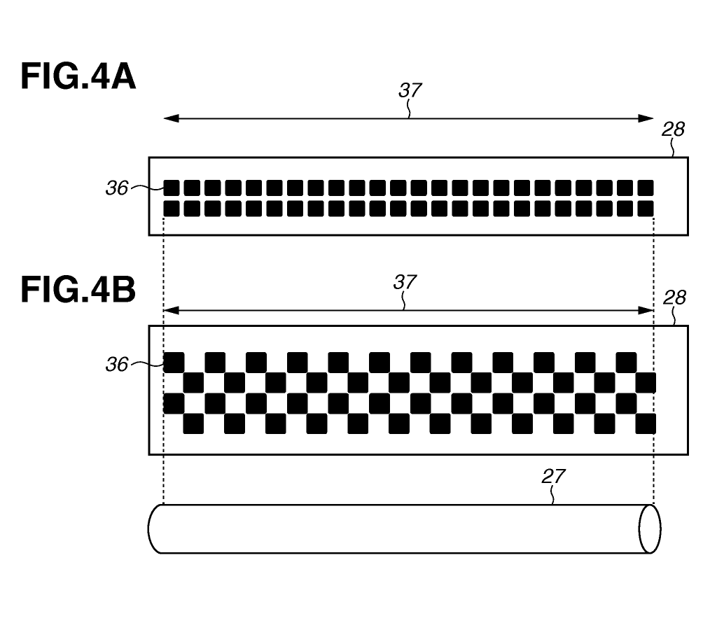

FIG. 4(a) and FIG. 4(b) are schematic diagrams showing the state in which a plurality of light emitting units 36 is arranged on the exposure light source 28 on a long substrate. The arrow 37 shows the line direction in which the organic light-emitting elements are arranged. This line direction is the same as the direction of an axis in which the photo conductor 27 rotates.

FIG. 4(a) represents the form in which light-emitting units are arranged along the axis direction of the photo conductor. FIG. 4(b) represents the form in which light-emitting units are alternately arranged in the line direction in each of the first line and the second line. In the first line and the second line, the units are arranged in different places in the row direction.

In the first line, a plurality of light emitting units is arranged at intervals. The second line contains light emitting units in the places corresponding to the intervals of light emitting units in the first line. That is, a plurality of light emitting units is arranged at intervals in the row direction.

In other words, the arrangement in FIG. 4(b) can be referred to as, for example, the state having a lattice arrangement, the state having a zigzag lattice arrangement, a checked pattern or the state having a stagger.

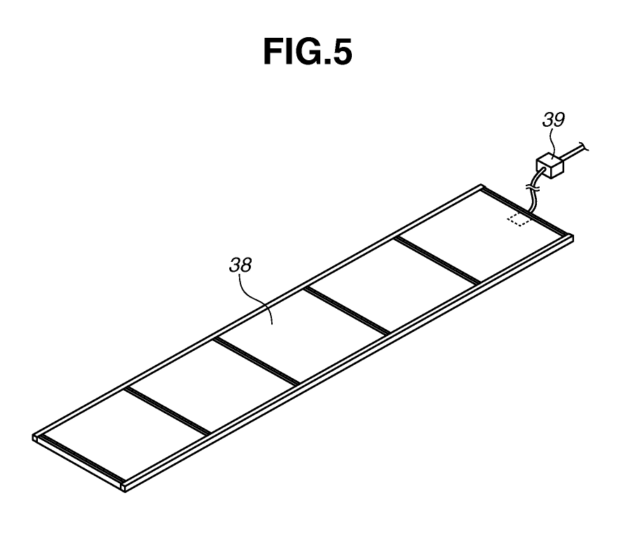

FIG. 5 is a schematic diagram of the lighting device according to the embodiment. The lighting device contains a substrate, organic light-emitting elements 38 and an AC/DC converter circuit 39. In addition, for example, a radiator plate, which is not shown, can be contained in the back side of a substrate surface on the side on which the organic light-emitting elements are put.

As described above, by driving a display device using the organic light-emitting element of the embodiment, a display which has an excellent image quality and is stable for a long period of time can be obtained.

### EXAMPLES

### Example 1

### Synthesis of Exemplified Compound B10

(1) NFCl-1 represented below is synthesized according to synthetic scheme shown in the following formula [8].

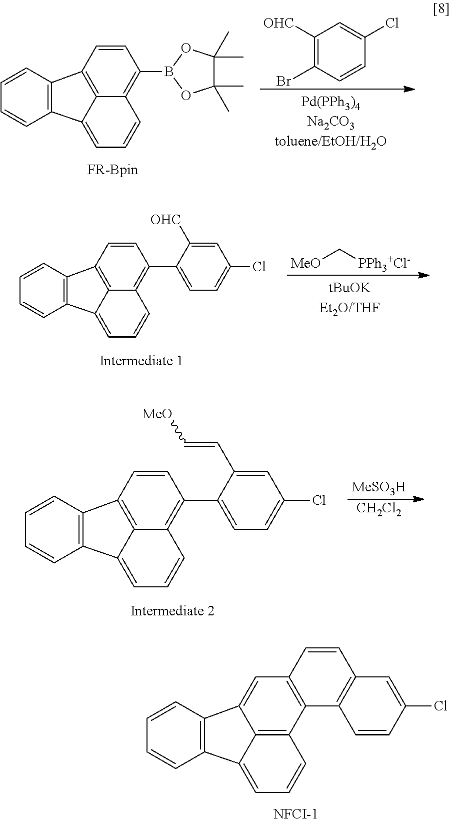

To a 200 mL recovery flask, the following reagents and solvents were placed.

2-bromo-5-chlorobenzaldehyde: 2.00 g (9.11 mmol)

FR-Bpin: 3.08 g (9.39 mmol)
Tetrakis(triphenylphosphine)palladium(0): 210 mg (0.18 mmol)

### Toluene: 60 mL

### Ethanol: 30 mL

Aqueous solution of 10 wt % Na₂CO₃: 30 mL

The resulting reaction solution was refluxed for 6 hours under heating and stirring in nitrogen gas. After the reaction ends, toluene and water were added to the reaction solution. The solution was stirred and an organic layer is separated therefrom. After the organic layer was washed by saturated sodium chloride solution, the organic layer was dried by sodium sulfate. The crude product was obtained from the organic layer by vacuum concentration. The crude product was refined by Silica-gel chromatography (eluent:toluene). And 2.84 g (yield: 92%) of the intermediate 1 was obtained by recrystallization in toluene and heptanes.

Next, to a 200 mL recovery flask filled with nitrogen, (Methoxymethyl) triphenylphosphonium chloride: 7.14 g (20.8 mmol), dehydrated diethyl ether: 36 mL was placed in room temperature. After 1 M THF solution of tert-buthoxy potassium: 20.8 (20.8 mmol) was added to the reaction solution with stirring, the reaction solution was stirred for 1 hour. Next, 2.84 g (8.33 mmol) of the intermediate 1 dissolved in THF 68 mL was added to the reaction solution. After stirring the reaction solution in room temperature for 2.5 hours, water was added to the reaction solution to stop the reaction. After, water layer was extracted twice using ethyl acetate, the organic layer was recovered, and the organic layer was washed by saturated sodium chloride solution and dried by sodium sulfate. The crude product was obtained by vacuum concentration. 2.92 g (yield 95%) of the intermediate 2 was obtained by refining the crude product with Silica-gel chromatography (eluent:heptane/chloroform=2/1).

To a 500 mL recovery flask filled with nitrogen, 2.92 g (8.67 mmol) of the intermediate 2 and dehydrated dichloromethane 100 mL were placed. After 10 drops of methanesulfonic acid was added to the reaction solution with stirring at room temperature, and further stirring for 30 minutes, methanol was added to the reaction solution to stop the reaction. 1.30 g of NFCl-1 was obtained by filtrated yellow crystal separated by above reaction. Identification of NFCl-1 obtained was conducted by the following method.

[MALDI-TOF-MS (Matrix Assisted Laser Desorption Ionization—Time Of Flight—Mass Spectrometry)]

Measured value: m/z=336.25, calculated value: C₂₄H₁₃Cl=336.07 [¹H-NMR (400 MHz, CDCl₃)]

δ 9.15 (d, 1H), 8.92 (d, 1H), 8.30 (s, 1H), 8.10-7.91 (m, 5H), 7.90-7.80 (m, 2H), 7.67 (dd, 1H), 7.44 (m, 2H).

(2) NFBpin-1 represented below was synthesized according to synthetic scheme shown in the following formula [9].

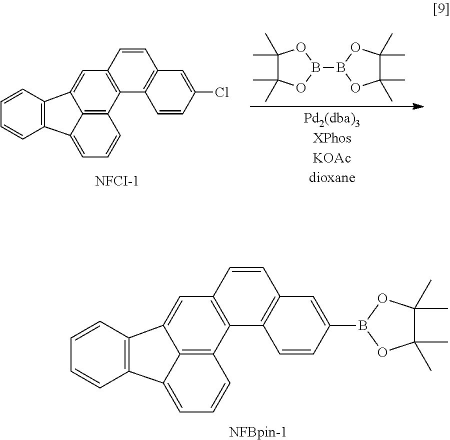

Specifically, to a 100 mL recovery flask filled with nitrogen, the following reagents and solvents were placed.

NFCl-1:800 mg (2.38 mmol)

Bis(pinacolato)diboron: 1.81 g (7.13 mmol)
Pd₂[dba]₃: 43.6 mg (0.0476 mmol)
XPhos: 90.8 mg (0.190 mmol)
Potassium acetate: 700 mg (7.13 mmol)
Dioxane (dehydrated): 24 mL

After the reaction solution was bubbled with nitrogen for 10 minutes, the reaction solution was heated and stirred at 110° C. for 4.5 hours under nitrogen. After the reaction solution was refrigerated to room temperature, toluene was added to dilute the reaction solution. After a salt generated by diluting was removed by filtration, a crude product was obtained by vacuum concentration. 907 mg (yield 89%) of NFBpin-1 was obtained by refining the crude product with Silica-gel chromatography (eluent:toluene/heptane=4/1)

(3) Synthesis of exemplified compound B10 was conducted according to synthetic scheme shown in the following formula [10].

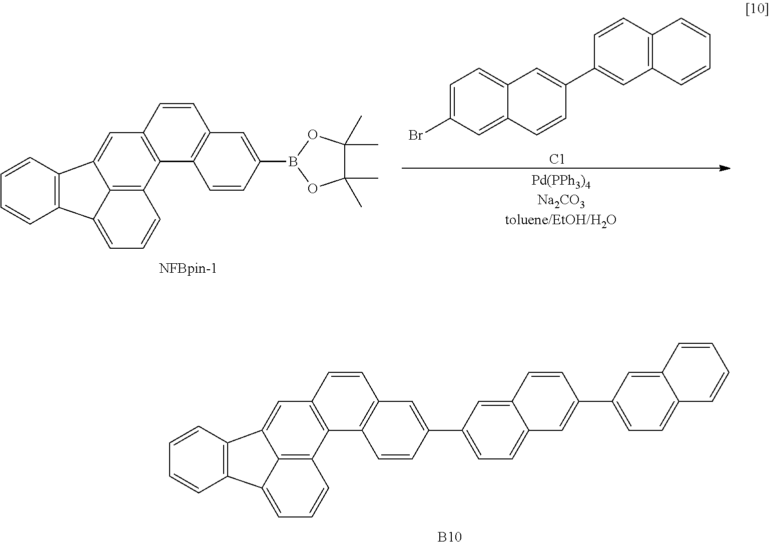

Specifically, to a 50 mL recovery flask, the following reagents and solvents were placed.

NFBpin-1:350 mg (0.817 mmol)

C1:329 mg (0.817 mmol)
Tetrakis(triphenylphosphine)palladium(0): 19 mg (16.3 □mol)

### Toluene: 8 mL

### Ethanol: 4 mL

Aqueous solution of 10 wt % Na₂CO₃: 4 mL

The resulting reaction solution was refluxed for 3 hours under heating and stirring in nitrogen gas. After the reaction ends, water was added to the reaction solution. The reaction solution was stirred and was filtrated. A crystal was separated by above operation and a crude product was obtained by performing washing using water and ethanol in this order. After the crude product was heated and dissolved in chlorobenzene, the crude product was hot gel filtrated by using a few Silica-gel. After the filtrate was concentrated in vacuum, conducting recrystallization using chlorobenzene and obtaining a crystal. The crystal was dried in vacuum at 150° C. and then 310 mg (yield 69%) of the exemplified compound B10. 44 mg of the exemplified compound B10 which is high purity was obtained by purification method by sublimation under 1*10⁻⁴ Pa and 370° C.

Identification of the compound obtained was conducted by mass spectrometry.

[MALDI-TOF-MS]

Measured value: m/z=554.39, calculated value: C₄₄H₂₆=554.20

### Example 2

### Synthesis of the Exemplified Compound A10

The exemplified compound A10 was obtained as Example 2 except using an organic compound C2 represented by the following structure instead of the organic compound C1 in Example 1.

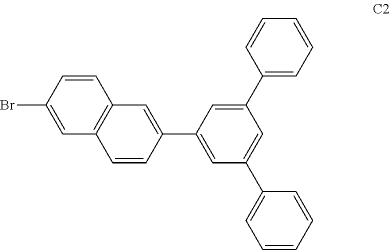 [SMILES: Brc1ccc2cc(-c3cc(-c4ccccc4)cc(-c4ccccc4)c3)ccc2c1]

Identification of the compound obtained was conducted by mass spectrometry.

[MALDI-TOF-MS]

Measured value: m/z=656.71, calculated value: C₅₂H₃₂=656.81

### Example 3

### Synthesis of the Exemplified Compound A13

The exemplified compound A13 was obtained as Example 3 except using an organic compound C3 represented by the following structure instead of the organic compound C1 in Example 1.

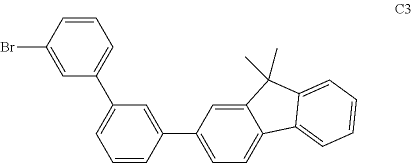 [SMILES: CC1(C)c2ccccc2-c2ccc(-c3cccc(-c4cccc(Br)c4)c3)cc21]

Identification of the compound obtained was conducted by mass spectrometry.

[MALDI-TOF-MS]

Measured value: m/z=646.31, calculated value: C₅₁H₃₄=646.82

### Example 4

### Synthesis of the Exemplified Compound B12

The exemplified compound B12 was obtained as Example 4 except using an organic compound C4 represented by the following structure instead of the organic compound C1 in Example 1.

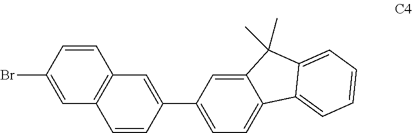 [SMILES: CC1(C)c2ccccc2-c2ccc(-c3ccc4cc(Br)ccc4c3)cc21]

Identification of the compound obtained was conducted by mass spectrometry.

[MALDI-TOF-MS]

Measured value: m/z=620.83, calculated value: C₄₉H₃₂=620.78

### Example 5

### Synthesis of the Exemplified Compound B13

The exemplified compound B13 was obtained as Example 5 except using an organic compound C5 represented by the following structure instead of the organic compound C1 in Example 1.

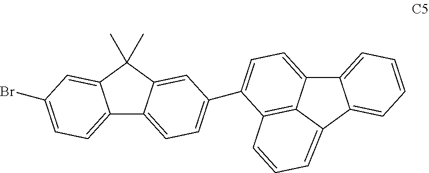 [SMILES: CC1(C)c2cc(Br)ccc2-c2ccc(-c3ccc4c5c(cccc35)-c3ccccc3-4)cc21]

Identification of the compound obtained was conducted by mass spectrometry.

[MALDI-TOF-MS]

Measured value: m/z=694.73, calculated value: C₅₅H₃₄=694.86

### Example 6

### Synthesis of the Exemplified Compound B16

The exemplified compound B16 was obtained as Example 6 except using an organic compound C6 represented by the following structure instead of the organic compound C1 in Example 1.

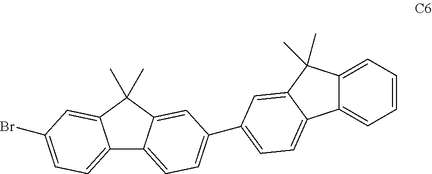 [SMILES: CC1(C)c2ccccc2-c2ccc(-c3ccc4c(c3)C(C)(C)c3cc(Br)ccc3-4)cc21]

Identification of the compound obtained was conducted by mass spectrometry.

[MALDI-TOF-MS]

Measured value: m/z=686.53, calculated value: C₅₄H₃₈=686.88

### Comparative Example 1

### Synthesis of the Reference Compound D1

The reference compound D1 was synthesized according to the synthetic scheme shown in the following formula [11]. And then, the reference compound D1 which is high purity was obtained by purification method by sublimation. It is noted that operations of reactions and purification were the same as Example 1.

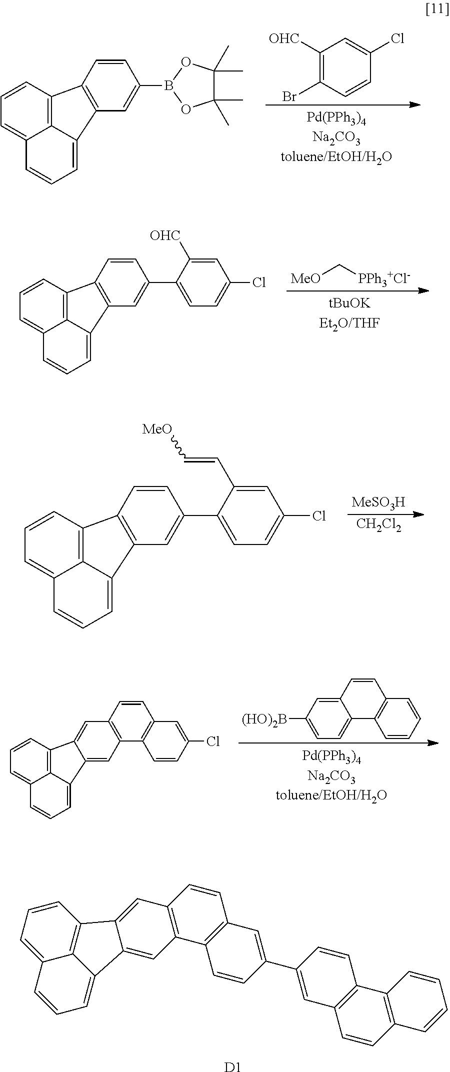

Identification of the compound obtained was conducted by mass spectrometry.

[MALDI-TOF-MS]

Measured value: m/z=616.51, calculated value: C₄₉H₂₈=616.75

### Comparative Example 2

### Synthesis of the Reference Compound D2

The reference compound D2 was synthesized according to the synthetic scheme shown in the following formula [12]. And then, the reference compound D2 which is high purity was obtained by purification method by sublimation. It is noted that operations of reactions and purification were the same as Example 1.

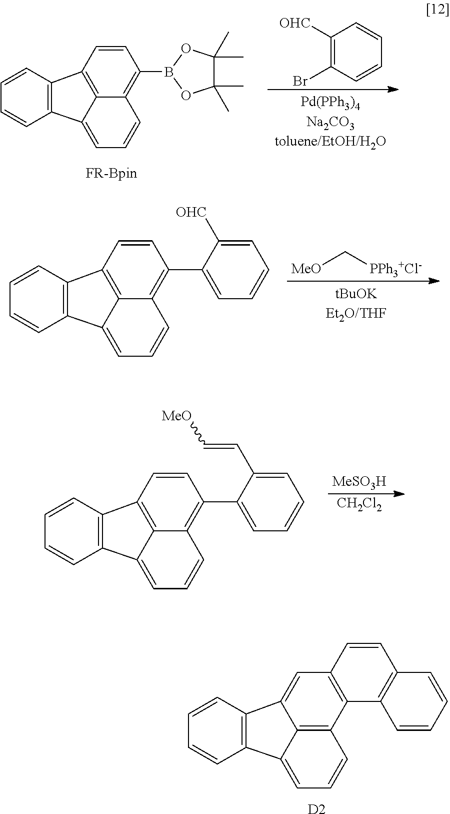

Identification of the compound obtained was conducted by mass spectrometry.

[MALDI-TOF-MS]

Measured value: m/z=302.62, calculated value: C₂₄H₁₄=302.37

Herein, the reference compound D1 is the naphtho[2,1-b]fluoranthene compound described in PTL 1 and the reference compound D2 is the naphtho[2,1-b]fluoranthene compound described in PTL 2.

### Example 7

In this example, an organic light-emitting device in which an anode, a hole transporting layer, an electron blocking layer, a light-emitting layer, a hole blocking layer, an electron transporting layer, an electron injection layer and a cathode were sequentially formed on a substrate was produced.

First, an ITO film was formed on a glass substrate, and an ITO electrode (anode) was formed by carrying out desired patterning processing. At this time, the film thickness of the ITO electrode was 100 nm. As described above, the substrate in which the ITO electrode was formed was used as an ITO substrate in the following step.

The organic compound layers and electrode layer were formed on the above-mentioned ITO substrate shown in Table 3 given below. It is noted that at this time, the electrodes facing each other (metal electrode layer, cathode) were formed so that the electrode area thereof would be 3 mm².

TABLE 3

| | | FILM |
|---|---|---|
| | | THICKNESS |
| | MATERIAL | (nm) |
| HOLE TRASPORTING | HT2 | 40 |
| LAYER | | |
| ELECTRON BLOCKING | HT8 | 10 |
| LAYER | | |
| LIGHT-EMITTING LAYER | EM12 (HOST) | 30 |
| | RD1 (GUEST) | |
| | EM12:RD1 = 99:1 | |
| | (WEIGHT RATIO) | |
| HOLE BLOCKING LAYER | EXEMPLIFIED | 10 |
| | COMPOUND B10 | |
| ELECTRON | ET2 | 30 |
| TRANSPORTING LAYER | | |
| ELECTRON INJECTION | LiF | 1 |
| LAYER | | |
| METAL ELECTRODE | A1 | 100 |
| LAYER | | |

In the organic light-emitting device, a current density of 100 mA/cm² was applied to the ITO electrode as an anode and the Al electrode as a cathode. The emission efficiency was 3.8 cd/A and the applied voltage was 3.9 V. The red light from the organic light-emitting device was observed.

The life time of the organic light-emitting device of which the luminosity decreases 1.5% in case of driving at 250 mA/cm² of current density was measured to evaluate the stability of the organic light-emitting device. And then the life time was 150 hours.

The characteristic of current and voltage was measured by a micro ammeter 4140B made by Hewlett-Packard. The luminosity of the organic light-emitting device was measured by BM7 made by TOPCON.

### Example 8 to 15 and Comparative Example 3 to 5

In these examples, organic light-emitting devices were produced in the same manner as ins Example 7 except changing a host material and a guest material of a light-emitting layer and a material of a hole blocking layer. The organic light-emitting devices were evaluated in the same manner as Example 7. The result was shown in Table 4.

TABLE 4

| | | | | | | LIFE |
|---|---|---|---|---|---|---|
| | | | | | | TIME TO |
| | | | | | | LUMINOUS |
| | | | HOLE | LUMINOUS | | DECREASING |
| | | | BLOCKING | EFFICIENCY | VOLTAGE | 1.5% |
| | HOST | GUEST | LAYER | (cd/A) | (V) | (h) |
| Example | EM12 | RD1 | EXEMPLIFIED | 4.2 | 4.2 | 150 |
| 8 | | | COMPOUND | | | |
| | | | A10 | | | |
| Example | EM12 | RD2 | EXEMPLIFIED | 4.3 | 4.2 | 130 |
| 9 | | | COMPOUND | | | |
| | | | A13 | | | |
| Example | EM12 | RD1 | EXEMPLIFIED | 4.0 | 4.0 | 150 |
| 10 | | | COMPOUND | | | |
| | | | B12 | | | |
| Example | EM12 | RD2 | EXEMPLIFIED | 3.9 | 4.0 | 120 |
| 11 | | | COMPOUND | | | |
| | | | B13 | | | |
| Example | EM12 | RD2 | EXEMPLIFIED | 4.0 | 3.9 | 120 |
| 12 | | | COMPOUND | | | |
| | | | B14 | | | |
| Example | EM12 | RD1 | EXEMPLIFIED | 4.1 | 3.9 | 150 |
| 13 | | | COMPOUND | | | |
| | | | B16 | | | |
| Example | EXEMPLIFIED | RD1 | ET11 | 3.6 | 4.2 | 120 |
| 14 | COMPOUND | | | | | |
| | A13 | | | | | |
| reference | EM12 | RD1 | REFERENCE | 3.1 | 4.6 | 100 |
| example | | | COMPOUND | | | |
| 3 | | | D2 | | | |
| reference | EM12 | RD1 | REFERENCE | 1.4 | 5.2 | 50 |
| example | | | COMPOUND | | | |
| 4 | | | D2 | | | |
| Example | EM3 | GD4 | EXEMPLIFIED | 4.5 | 4.5 | 100 |
| 15 | | | COMPOUND | | | |
| | | | A10 | | | |
| reference | EM3 | GD4 | REFERENCE | 3.4 | 5.3 | 50 |
| example | | | COMPOUND | | | |
| 5 | | | D1 | | | |

As described above, an organic light-emitting device including a hole blocking layer comprising the naphtho[2,1-b]fluoranthene compound according to the embodiment is a device which has low driving voltage, high luminous efficiency and long life time. It is because S1 energy of the naphtho[2,1-b]fluoranthene compound according to the embodiment is sufficiently high, properties of blocking exciton and hole are high, and property of electron injection is high. And in case of using the naphtho[2,1-b]fluoranthene compound according to aspects of the present invention as a host material of a light-emitting layer, the obtained organic light-emitting device also has low driving voltage, high luminous efficiency and long life time.

### Example 16

In this example, an organic light-emitting device having an optical resonator structure was produced according to the following method.

100 nm thickness of AlNd electrode as reflecting anode was formed by a sputtering method on a glass substrate as supporting body.

In addition, 80 nm thickness of ITO electrode as transmissive anode was formed by the sputtering method. 1.5 □□m (micro meter) of element separate film was formed around part of the anode and was provided an opening with the radius of 3 mm.

After the substrate and the electrode were washed by ultrasonic in acetone and isopropyl alcohol (IPA) in this order, the substrate and the electrode were boiled and washed in IPA and dried. In addition, UV wash was conducted to the substrate and the electrode.

After organic compound layers shown in the following Table 5 were formed sequentially by vacuum evaporation in a vacuum chamber at 1*10⁻⁵ Pa, IZO as a cathode was formed by sputtering method to obtain 30 nm thickness of a transmissive electrode. After being formed these, sealing was performed under nitrogen gas. From the above, the organic light-emitting device was obtained.

TABLE 5

| | | THICKNESS |
|---|---|---|
| | MATERIAL | (nm) |
| HOLE TRANSPORTING LAYER | HT2 | 185 |
| ELECTRON BLOCKING LAYER | HT8 | 10 |
| LIGHT-EMITTING LAYER | EM12 (HOST) | 30 |
| | RD1 (GUEST) | |
| | EM12:RD1 = 99:1 | |
| | (WEIGHT RATIO) | |
| HOLE BLOCKING LAYER | EXEMPLIFIED | 10 |
| | COMPOUND B10 | |
| ELECTRON | ET2:LiF = 80:20 | 70 |
| INJECTION/TRANSPORTING | | |
| LAYER | | |

In the organic light-emitting device, a voltage of 100 mA/cm² was applied to the ITO electrode as an anode and the IZO electrode as a cathode. The emission efficiency was 10.6 cd/A. The red light with a luminosity of 2000 cd/m² from the organic light-emitting device was observed.

From the above, the naphtho[2,1-b]fluoranthene compound according to aspects of the present invention is able to suppress exciton leakage from a light-emitting layer since S1 energy of the organic compound is sufficiently large. Also, the organic compound is able to suppress hole leakage from a light-emitting layer since the HOMO level of the organic compound is deep and the organic compound has a high property of electron injection from an electron injection layer or an electron transporting layer since LUMO level of the organic compound is deep. The structure of the organic compound is able to suppress stacking of the molecules since a structure of the naphtho[2,1-b]fluoranthene backbone, main backbone of the organic compound, is non-planar.

Therefore, in a case where the naphtho[2,1-b]fluoranthene compound according to aspects of the present invention is used as constitution materials for organic light-emitting devices such as materials for a hole blocking layer, the obtained organic light-emitting device has a low driving voltage, a high luminous efficiency and a long life time.

According to aspects of the present invention, the naphtho[2,1-b]fluoranthene compound in which molecular stacking is suppressed and the deep HOMO level as well as the deep LUMO level are ensured can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-232194, filed Nov. 14, 2014, which is hereby incorporated by reference herein in its entirety.

What is claimed is:

1. A naphtho[2,1-b]fluoranthene compound represented by Formula [1]: 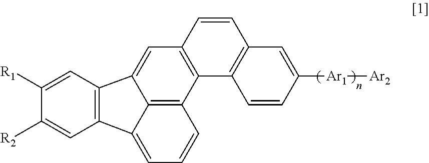
wherein R₁ and R₂ are respectively selected from the group consisting of a hydrogen atom, an alkyl group having 1 to 4 carbon atoms, an alkoxy group having 1 or 2 carbon atom, a halogen atom and a cyano group;
wherein Ar₁ denotes an aromatic hydrocarbon having 6 to 24 carbon atoms and may be substituted by a substituent selected from the group consisting of an alkyl group having 1 to 4 carbon atom, an alkoxy group having 1 or 2 carbon atom, a halogen atom and a cyano group;
wherein Ar₂ denotes an aromatic hydrocarbon having 6 to 24 carbon atoms and may be substituted by a substituent selected from the group consisting of an alkyl group having 1 to 4 carbon atom, an alkoxy group having 1 or 2 carbon atom, a halogen atom and a cyano group; and.
wherein n is integer of 0 to 3, and in a case where n is 2 or more, each Ar₁ may be the same or different.

2. The naphtho[2,1-b]fluoranthene compound according to claim 1,
wherein both of the R1 and the R2 are hydrogen atom.

3. The naphtho[2,1-b]fluoranthene compound according to claim 1,
wherein the aromatic hydrocarbon represented by Ar₁ is one of a phenylene group, a naphthalene-diyl group, a fluorene-diyl group, a phenanthrene-diyl group, a chrysene-diyl group, a biphenyl-diyl group, a terphenyl-diyl group, a fluoranthene-diyl group and a triphenylene-diyl group; and
wherein the aromatic hydrocarbon represented by Ar₂ is one of a phenyl group, a naphthyl group, a fluorenyl group, a phenanthrenyl group, a chrysenyl group, a biphenylyl group, a terphenyl group, a fluoranthenyl group and a triphenyl group.

4. The naphtho[2,1-b]fluoranthene compound according to claim 1,
wherein n denotes 1.

5. The naphtho[2,1-b]fluoranthene compound according to claim 1,
wherein the Ar₁ is selected from the group consisting of a phenylene group bound to a naphtho[2,1-b]fluoranthene backbone with Ar₂ at the 1-position and the 4-position, a naphthalene-diyl group bound to a naphtho[2,1-b]fluoranthene backbone with Ar₂ at the 2-position and the 6-position, a 9,9′-dimethyl-fluorene-diyl group bound to a naphtho[2,1-b]fluoranthene backbone with Ar₂ at the 2-position and the 7-position, a phenathrene-diyl group bound to a naphtho[2,1-b]fluoranthene backbone with Ar₂ at the 2-position and the 7-position, a biphenyl-diyl group bound to a naphtho[2,1-b]fluoranthene backbone with Ar₂ at the 4-position and the 4′-position, and a terphenyl-diyl group bound to a naphtho[2,1-b]fluoranthene backbone with Ar₂ at the 4-position and the 4″-position; and
wherein the Ar₂ is selected from a group consisting of a phenyl group bound at the 1-position, a naphthyl group bound at the 2-position, a 9,9′-dimethyl-fluorenyl group bound at the 2-position, a phenanthrenyl group bound at the 2-position, a biphenylyl group bound at the 4-position, a terphenyl group bound at the 4-position, a fluoranthene group bound at the 3-position or the 8-position and triphenylene group bound at the 2-position.

6. An organic electronic device comprising:
a pair of electrodes; and
an organic compound layer placed between the pair of electrodes;
wherein the organic compound layer comprises the naphtha[2,1-b]fluoranthene compound according to claim 1.

7. An organic light-emitting device comprising:
an anode;
a cathode; and
a light-emitting layer placed between the anode and the cathode;
wherein the organic light-emitting device further comprises an organic compound layer placed between the cathode and the light-emitting layer; and
wherein the organic compound layer comprises the naphtha[2,1-b]fluoranthene compound according to claim 1.

8. The organic light-emitting device according to claim 7,
wherein the organic compound layer contacts the light-emitting layer.

9. The organic light-emitting device according to claim 7,
wherein a HOMO energy level of the organic compound layer is 0.3 eV higher than that of the light-emitting layer.

10. The organic light-emitting device according to claim 7, further comprising a second organic compound layer placed between the cathode and the organic compound layer,
wherein a difference between a LUMO energy level of the organic compound layer and that of the second organic compound layer is 0.5 eV or less.

11. A display apparatus comprising a plurality of pixels,
wherein at least one of the plurality of pixels comprises the organic light-emitting device according to claim 7 and an active element connected to the organic light-emitting device.

12. The display apparatus according to claim 11,
wherein the display apparatus includes a detached space between one pixel and another pixel each included in the plurality of pixels, and the organic compound layer is placed in succession to the one and the another pixels and the detached space.

13. A lighting apparatus comprising:
the organic light-emitting device according to claim 7; and
an AC/DC convertor connected to the organic light-emitting device.

14. A lighting apparatus comprising:
the organic light-emitting device according to claim 7; and
a heat dissipation unit which dissipates heat out of the apparatus.

15. An image forming apparatus comprising:
a photosensitive member;
an exposure unit which exposes the photosensitive member;
a charging unit which charges the photosensitive member; and
a developing unit which applies developer to a surface of the photosensitive member,
wherein the exposure unit includes the organic light-emitting device according to claim 7.

16. An exposure apparatus which exposes a photosensitive member,
wherein the exposure apparatus comprises a plurality of light-emitting devices according to claim 7, and
wherein the plurality of the light-emitting devices are arranged in a line in a longitudinal direction of the photosensitive member.
